(19) United States
(12) Patent Application Publication
(10) Pub. No.: US 2014/0378380 A1
(43) Pub. Date: Dec. 25, 2014

(54) USE OF UNACYLATED GHRELIN, FRAGMENTS AND ANALOGS THEREOF AS ANTIOXIDANT

(71) Applicant: Alizé Pharma SAS, Ecully (FR)

(72) Inventors: Maria Felice Brizzi, Turin (IT); Ezio Ghigo, Turin (IT)

(21) Appl. No.: 14/310,089

(22) Filed: Jun. 20, 2014

Related U.S. Application Data
(60) Provisional application No. 61/837,723, filed on Jun. 21, 2013.

(51) Int. Cl. A61K 38/25

(52) U.S. Cl. CPC A61K 38/25
USPC 514/11.2; 435/374

(57) ABSTRACT

The present invention relates to methods for protecting a subject, tissues and/or organs from a subject against oxidative stress-induced damage, such as but not limited to, oxidative stress-induced tissue damage. The method comprises administering an effective amount of unacylated ghrelin, a fragment thereof, an analog thereof and/or pharmaceutically acceptable salts thereof to the subject.

### CROSS-REFERENCE TO RELATED APPLICATIONS

This application claims the benefit of and priority to U.S. provisional patent application No. 61/837,723; filed Jun. 21, 2013, the content of which is herein incorporated in its entirety by reference.

### FIELD OF TECHNOLOGY

The present invention relates to the field of oxidative stress-induced damage and the development of compositions and methods to protect against oxidative stress-induced damage, to reduce oxidative stress-induced damage and to improve resistance to oxidative stress-induced damage. The present invention also relates to the use of unacylated ghrelin, fragments and analogs thereof, in order to protect against oxidative stress-induced damage, to reduce oxidative stress-induced damage and to improve resistance to oxidative stress-induced damage.

### BACKGROUND INFORMATION

Ghrelin (also referred as “acylated ghrelin” or abbreviated as “AG”) is a 28 amino acid peptide, purified and identified from rat stomach and characterized by the presence of an n-octanoyl modification on the Ser3 residue¹. Acylation of ghrelin is catalyzed by the enzyme ghrelin O-acyl transferase (GOAT). Ghrelin is the endogenous ligand of the growth hormone (GH) secretagogue receptor (GHSR-1a)^{2,3} and is now mostly recognized as a potent orexigenic factor stimulating food intake and modulating energy expenditure^{4,5,6}. At the peripheral level, ghrelin exerts probably its major physiological action regulating glucose and lipid metabolism⁷. In fact, ghrelin has a diabetogenic action⁸ and suppresses glucose-stimulated insulin secretion and deteriorates glucose tolerance⁹.

Unacylated ghrelin (also referred as “des-acyl ghrelin” or abbreviated as “UAG”), is the non-acylated form of ghrelin. Its concentration in plasma and tissue is higher compared to ghrelin. UAG has long been considered as a product with no physiological role as it fails to bind the only known ghrelin receptor GHSR-1a at physiological concentrations and has no physiological effect on GH secretion¹⁰. However, UAG is a biologically active peptide⁴⁹. It has been shown to prevent the hyperglycemic effects of ghrelin when administered concomitantly in healthy volunteers (as reported in U.S. Pat. No. 7,825,090, herein incorporated in its entirety by reference). This initial observation is supported by several reports on the anti-diabetogenic potential of UAG¹²⁻¹⁶. The anti-diabetogenic effects and ghrelin-antagonizing effects of UAG, fragments and analogs thereof have been reported in U.S. Pat. No. 7,485,620; U.S. Pat. No. 7,666,833; U.S. Pat. No. 8,071,368; U.S. Pat. No. 8,222,217; U.S. Pat. No. 8,318,664; U.S. Pat. No. 8,476,408 and in U.S. Patent Application 2010/0016226, U.S. Patent Application 2013/0157936, WO 2009/150214 and WO 2013/088241 which are all in their entirety incorporated herein by reference.

AG and UAG have been shown to exert effects on muscle cell and vascular cell differentiation through a common receptor^{18,19}. However, several studies have shown that UAG and AG exhibit opposing metabolic actions^{20,21,49}. Similarly, UAG and AG induce different biological responses in neonatal mouse and rat cardiomyocytes²² and only UAG protects endothelial progenitor cells (EPCs) from oxidative stress by avoiding reactive oxygen species (ROS) generation^{23,24,50} as reported in U.S. Patent Application 2010/0016226 and WO 2009/150214.

Oxidative stress plays a major role in tissue damage and is important in the development and progression of several conditions and diseases¹⁷. For example, oxidative stress is suspected to be significant in neurodegenerative diseases such as Lou Gehrig's disease, Parkinson's disease, Alzheimer's disease, and Huntington's disease. Cumulative oxidative stress with disrupted mitochondrial respiration and mitochondrial damage has been associated with Alzheimer's disease, Parkinson's disease, and other neurodegenerative diseases. Oxidative stress is also thought to be linked to certain cardiovascular disease.

Oxidative stress further plays a role in the ischemic cascade due to oxygen reperfusion injury following hypoxia (i.e., reperfusion injury). Oxidative stress has also been implicated in chronic fatigue syndrome and shown to contribute to tissue injury following irradiation and hyperoxia, as well as in diabetes.

Oxidative stress is present in peripheral arterial disease (PAD) which is a widespread condition caused by atherosclerosis of the peripheral arteries²⁵. Although surgical or endovascular intervention remains the standard therapy to improve blood flow²⁶, even after successful revascularisation, most patients complain of persistent or recurring symptoms²⁷. Changes in local oxygen availability in PAD result in increased numbers of dysfunctional mitochondria^{28,29}. Defective mitochondrial electron transfer chain and increased ROS generation are important determinants of oxidative stress-induced damage and impaired cellular functions³⁰⁻³³ that ultimately lead to muscle damage³⁴. Interestingly, superoxide dismutase-2 (SOD-2), the initial line of defense against ROS in the mitochondria, is deficient in PAD muscles²⁸. Consistently, antioxidant administration ameliorates skeletal muscle mitochondrial dysfunction and functional recovery in humans³⁵.

In an aging population with an increasingly high incidence of metabolic diseases, new treatment options for circumventing the damages caused by oxidative stress represents a major unmet need. The earlier observation that UAG, fragments and analogs thereof protect endothelial progenitor cells (EPCs) from diabetes-associated oxidative stress by avoiding AGE-induced ROS generation^{23,24,50} as led to evaluate the protective effect of UAG as an antioxidant.

### SUMMARY OF THE INVENTION

According to one aspect, the present invention relates to a method for protecting a subject against oxidative stress-induced damage, comprising administering an effective amount of unacylated ghrelin, a fragment thereof, an analog thereof and/or pharmaceutically acceptable salts thereof to the subject.

According to another aspect, the present invention relates to a method for reducing oxidative stress-induced damage in a subject, comprising administering an effective amount of unacylated ghrelin, a fragment thereof, an analog thereof and/or pharmaceutically acceptable salts thereof to the subject.

According to another aspect, the present invention relates to a method for improving tolerance to oxidative stress-induced damage in a subject, comprising administering an effective amount of unacylated ghrelin, a fragment thereof, an analog thereof and/or pharmaceutically acceptable salts thereof to the subject.

According to another aspect, the present invention relates to a method for ameliorating an oxidative stress-associated condition in a subject, comprising administering an effective amount of unacylated ghrelin, a fragment thereof, an analog thereof and/or pharmaceutically acceptable salts thereof to the subject.

According to another aspect, the present invention relates to the use of an effective amount of unacylated ghrelin, a fragment thereof, an analog thereof and/or pharmaceutically acceptable salts thereof for protecting a subject against oxidative stress-induced damage.

According to another aspect, the present invention relates to the use of an effective amount of unacylated ghrelin, a fragment thereof, an analog thereof and/or pharmaceutically acceptable salts thereof for reducing oxidative stress-induced damage in a subject.

According to another aspect, the present invention relates to the use of an effective amount of unacylated ghrelin, a fragment thereof, an analog thereof and/or pharmaceutically acceptable salts thereof for improving tolerance to oxidative stress in a subject.

According to another aspect, the present invention relates to a method for protecting a tissue and/or an organ against oxidative stress-induced damage, comprising contacting the tissue and/or the organ with an effective amount of unacylated ghrelin, a fragment thereof, an analog thereof and/or pharmaceutically acceptable salts thereof.

According to another aspect, the present invention relates to a method for reducing oxidative stress-induced damage in a tissue and/or an organ, comprising contacting the tissue and/or the organ with an effective amount of unacylated ghrelin, a fragment thereof, an analog thereof and/or pharmaceutically acceptable salts thereof.

According to another aspect, the present invention relates to a method for improving resistance to oxidative stress in a tissue and/or an organ, comprising contacting the tissue and/or the organ with an effective amount of unacylated ghrelin, a fragment thereof, an analog thereof and/or pharmaceutically acceptable salts thereof.

According to another aspect, the present invention relates to the use of an effective amount of unacylated ghrelin, a fragment thereof, an analog thereof and/or pharmaceutically acceptable salts thereof for protecting a tissue and/or an organ against oxidative stress-induce damage.

According to another aspect, the present invention relates to the use of an effective amount of unacylated ghrelin, a fragment thereof, an analog thereof and/or pharmaceutically acceptable salts thereof for reducing oxidative stress-induce damage in a tissue and/or an organ.

According to another aspect, the present invention relates to the use of an effective amount of unacylated ghrelin, a fragment thereof, an analog thereof and/or pharmaceutically acceptable salts thereof for improving resistance to oxidative stress in a tissue and/or an organ.

According to another aspect, the present invention relates to a method for protecting a population of cells against oxidative stress-induced damage, comprising contacting the population of cells with an effective amount of unacylated ghrelin, a fragment thereof, an analog thereof and/or pharmaceutically acceptable salts thereof.

According to another aspect, the present invention relates to a method for reducing oxidative stress-induced damage in a population of cells, comprising contacting the population of cells with an effective amount of unacylated ghrelin, a fragment thereof, an analog thereof and/or pharmaceutically acceptable salts thereof.

According to another aspect, the present invention relates to a method for improving resistance against oxidative stress in a population of cells, comprising contacting the population of cells with an effective amount of unacylated ghrelin, a fragment thereof, an analog thereof and/or pharmaceutically acceptable salts thereof.

According to another aspect, the present invention relates to the use of an effective amount of unacylated ghrelin, a fragment thereof, an analog thereof and/or pharmaceutically acceptable salts thereof for protection of a population of cells against oxidative stress-induced damage.

According to another aspect, the present invention relates to the use of an effective amount of unacylated ghrelin, a fragment thereof, an analog thereof and/or pharmaceutically acceptable salts thereof for reduction of oxidative stress-induced damage in a population of cells.

According to another aspect, the present invention relates to the use of an effective amount of unacylated ghrelin, a fragment thereof, an analog thereof and/or pharmaceutically acceptable salts thereof for improving resistance to oxidative stress in a population of cells.

According to another aspect, the present invention relates to a method for improving repair of a tissue and/or an organ in a subject, comprising administering an effective amount of unacylated ghrelin, a fragment thereof, an analog thereof and/or pharmaceutically acceptable salts thereof to the subject.

According to another aspect, the present invention relates to a method for reducing oxidative stress-induced damage in a tissue and/or an organ of a subject, comprising administering an effective amount of unacylated ghrelin, a fragment thereof, an analog thereof and/or pharmaceutically acceptable salts thereof to the subject.

According to another aspect, the present invention relates to a the use of an effective amount of unacylated ghrelin, a fragment thereof, an analog thereof and/or pharmaceutically acceptable salts thereof for improving repair of a tissue and/or an organ in a subject.

According to another aspect, the present invention relates to the use of an effective amount of unacylated ghrelin, a fragment thereof, an analog thereof and/or pharmaceutically acceptable salts thereof for reducing oxidative stress-induced damage in a tissue and/or an organ of a subject.

According to another aspect, the present invention relates to a method for reducing oxidative stress-induced damage in a population of muscle cells, comprising contacting the population of skeletal muscle cells with an effective amount of unacylated ghrelin, a fragment thereof, an analog thereof and/or pharmaceutically acceptable salts thereof.

According to another aspect, the present invention relates to the use of an effective amount of unacylated ghrelin, a fragment thereof, an analog thereof and/or pharmaceutically acceptable salts thereof for reducing oxidative damage in a population of muscle cells.

According to another aspect, the present invention relates to an isolated unacylated ghrelin peptide, fragment thereof or analog thereof and/or pharmaceutically acceptable salts thereof for use in therapy for reducing oxidative stress-induced damage in a subject.

According to another aspect, the present invention relates to a method for modulating cellular levels of superoxide dismutase-2 (SOD-2) in a subject, comprising administering an effective amount of unacylated ghrelin, a fragment thereof, an analog thereof and/or pharmaceutically acceptable salts thereof to the subject.

According to another aspect, the present invention relates to a method for modulating cellular levels of superoxide dismutase-2 (SOD-2) in a population of cells, comprising contacting the population of cells with an effective amount of unacylated ghrelin, a fragment thereof, an analog thereof and/or pharmaceutically acceptable salts thereof.

According to another aspect, the present invention relates to a method for modulating cellular levels of superoxide dismutase-2 (SOD-2) in a tissue, comprising contacting the tissue with an effective amount of unacylated ghrelin, a fragment thereof, an analog thereof and/or pharmaceutically acceptable salts thereof.

According to another aspect, the present invention relates to a method for modulating cellular levels of superoxide dismutase-2 (SOD-2) in an organ, comprising contacting the organ with an effective amount of unacylated ghrelin, a fragment thereof, an analog thereof and/or pharmaceutically acceptable salts thereof.

According to another aspect, the present invention relates to the use of an effective amount of unacylated ghrelin, a fragment thereof, an analog thereof and/or pharmaceutically acceptable salts thereof to prevent reperfusion injury in an ischemic subject.

According to another aspect, the present invention relates to the use of an effective amount of unacylated ghrelin, a fragment thereof, an analog thereof and/or pharmaceutically acceptable salts thereof to prevent reperfusion injury in a cardiac ischemic subject.

### BRIEF DESCRIPTION OF THE FIGURES

FIGS. 1A to 1E illustrate the protective effect of UAG on ischemia-mediated functional impairment in skeletal muscle. In FIG. 1A, foot damage score was evaluated for the indicated times. In FIG. 1B, the number of vessels in ischemic (ih) and normo-perfused (nh) gastrocnemius muscles of each group of animals was evaluated. In FIG. 1C, sections of ischemic and normo-perfused (normal) muscles from UAG, AG and saline mice were stained. Insets show myofibers at higher magnification; black arrows indicate regenerating myofibers, characterized by central nucleus location. In FIG. 1D, the percentage of regenerating fibers was quantified and characterized by the presence of centrally located nucleus. In FIG. 1E, inflammatory cells in ischemic and normal muscles of UAG, AG and saline mice were quantified.

FIGS. 2A to 2F illustrate that UAG improves SMR and SC cell-cycle entry via p38/MAPK phosphorylation. In FIG. 2A, Pax-7+/MyoD+ cells in ischemic muscles were quantified. In FIG. 2B, the number of SCs from ischemic muscles of treated mice was calculated. In FIGS. 2C and 2D, cell extracts from SCs recovered from ischemic muscles were analyzed by Western blot for Pax-7 and phospho(p)-p38/MAPK (C) or for Myf5 and MyoD content (D). In FIG. 2E, sections of ischemic muscles recovered from treated mice were stained for myogenin and DAPI and myogenin+ cells in ischemic limb of treated mice were quantified. In FIG. 2F, the myogenin content was evaluated by Western blot in SCs from ischemic muscles of treated animals.

FIGS. 3A to 3E illustrate that UAG prevents ROS production in SCs by inducing SOD-2 expression. In FIG. 3A, TBARS were determined in gastrocnemius muscle of UAG, AG and saline mice. In FIG. 3B, ROS generation was evaluated by DCF-DA assay on SCs recovered from muscles of UAG, AG and saline mice. In FIG. 3C, SCs recovered from ischemic muscles of treated mice were subjected to Western blot normalized; SOD-2 content was evaluated. In FIG. 3D, representative sections of muscles recovered at day 7 after ischemia were stained (Pax-7, SOD-2 and DAPI staining) and Pax-7/SOD-2 positive cells in ischemic muscles of treated mice were quantified. FIG. 3E shows representative H&E stained sections of toxic damage induced by injection of 1% barium chloride (BaCl2) in gastrocnemius muscles of C57BL/6J mice.

FIGS. 4A to 4G illustrate the in vitro effects of UAG on primary SCs. In FIGS. 4A, 4B and 4C, SCs recovered from normoperfused muscles were subjected to in vitro ischemia in presence of the indicated stimuli. Cell extracts were analyzed by Western blot for Pax-7 and MyoD (A), for myogenin (B) and for pp38/MAPK content (C) by densitometry. In FIG. 4D, SCs subjected to in vitro ischemia and treated as indicated were analyzed by FACS analysis for PCNA expression. In FIG. 4E, FACS analysis indicates the percentage of SCs, treated as above, in the different cell-cycle phases. In FIG. 4F, ROS generation was evaluated by DCF-DA assay performed on SCs subjected to in vitro ischemia and treated as indicated. In FIG. 4G, SOD-2 content was analyzed by Western blot in SCs subjected to in vitro ischemia.

FIGS. 5A to 5J illustrate that UAG induces SC cell-cycle entry via SOD-2 and p38/MAPK phosphorylation. In FIG. 5A, SOD-2 content was evaluated in SCs transfected for 48 h with scramble or SOD-2 siRNA. In FIG. 5B, ROS generation was evaluated by DCF-DA assay performed on SCs treated as indicated. In FIG. 5C, FACS analysis indicates the percentage of SCs transfected with scramble or with SOD-2 siRNA in presence of UAG in the different cell-cycle phases. In FIG. 5D, SCs transfected with scramble or SOD-2 siRNA and stimulated with UAG were analyzed by Western blot for p-p38/MAPK content by densitometry. In FIG. 5E, FACS analysis indicates the percentage of SCs in the different cell-cycle phases following 24 h treatment with the indicated stimuli. In FIG. 5F, cell extracts from SCs treated as indicated were analyzed by Western blot for MyoD and myogenin content by densitometry. In FIG. 5G, SCs recovered from double KO mice were stimulated with saline, AG and UAG and subjected to in vitro ischemia. FACS analysis was performed to evaluate SC cell-cycle progression. In FIGS. 5H and 5I cell extracts from KO-derived SCs, treated as indicated and subjected to in vitro ischemia were analysed by western blot for Pax-7, MyoD, Myf5 and myogenin (H) and for p-p38/MAPK and SOD-2 (I) content by densitometry. In FIG. 5J, ROS generation was evaluated by DCF-DA assay performed on SCs derived from double KO mice treated as indicated.

FIGS. 6A to 6E illustrate that UAG induces SC cell-cycle entry by regulating miR-221/222 expression. In FIG. 6A, miR-221/222 expression was evaluated by qRT-PCR on SCs from ih and nh muscles of mice treated as indicated. In FIG. 6B, p57^{kip2} content was analyzed in SCs from ih and nh muscles by densitometry. In FIG. 6C, miR-221/222 expression was analyzed by qRT-PCR on SCs from nh muscles, subjected to in vitro ischemia and treated as indicated. In FIG. 6D, cell extracts from SCs treated as above were analyzed for p57^{kip2} content. In FIG. 6E, SCs were transfected with pmiR or pmiR-3′UTR p57^{kip2} luciferase constructs, treated as indicated and subjected to in vitro ischemia.

FIGS. 7A to 7E illustrate the in vivo effect of UAG on miR221-222 expression. In FIG. 7A, SOD-2 content in primary SCs, recovered from normo-perfused muscles and transfected for 48 h with the scramble or with the SOD-2 siRNA was analyzed. In FIG. 7B, miR-221/222 expression was evaluated by qRT-PCR on SCs silenced for SOD-2 and subjected to in vitro ischemia. FIG. 7C presents representative H&E stained sections of ischemic and normo-perfused (normal) muscles of mice injected with pre-miR negative control (neg ctrl) or with pre-miR221/222. Inset shows myofibers at higher magnification; black arrows indicate regenerating myofibers, characterized by central nucleus location. In FIG. 7D, foot damage score of treated mice was evaluated for the indicated times. In FIG. 7E, percentage of regenerating fibers in pre-miR neg ctrl or pre-miR-221/222-treated mice after ischemia was obtained.

FIG. 8 illustrates the protective effect of UAG and a fragment thereof on C2C12 mouse muscular cell line against oxidative stress. The effects of ROS production on C2C12 cells treated with UAG, UAG fragment (UAG (6-13)) and UAG cyclic fragment (UAG (6-13)cyclic) were assessed. Oxidative stress was induced by hypoxia (1% O₂), hyperglycemia (25 mM), advanced glycation end-products (AGEs) or H₂O₂.

### DETAILED DESCRIPTION

For ease of reference, the following abbreviations and designations are used herein throughout:

AG ghrelin or acylated ghrelin
UAG unacylated ghrelin or des-acyl ghrelin
UAG (6-13) unacylated ghrelin having residues 6 to 13 of SEQ ID NO: 1
GHSR growth hormone secretagogue receptor
ROS reactive oxygen species
RNS reactive nitrogen species
PAD peripheral arterial disease
SOD superoxide dismutase
SMR skeletal muscle regeneration
SC satellite cell
EPC endothelial progenitor cell
AGE advanced glycation end product
FACS florescence-activated cell sorting
e.g. for example

Unless defined otherwise, all technical and scientific terms used herein have the same meaning as commonly understood by one of skill in the art to which the invention pertains.

The invention defined in the present application stems from, but is not limited to, the unexpected findings by the inventors that UAG induces muscle regeneration after ischemia by reducing ROS-mediated muscle damage via a mechanism involving SOD-2 and miR221-222.

The data presented herein therefore provides the first evidence of the involvement of UAG, fragments thereof and analogs thereof in the primary enzymatic antioxidant defense against oxidative stress and ROS production and suggests the therapeutic potential of UAG, fragments thereof and analogs thereof in the treatment of conditions in which ROS scavenging and antioxidant efficiency is required.

### A) Unacylated Ghrelin, Fragments and Analogs Thereof

The expressions “unacylated ghrelin”, “des-acyl ghrelin” and the abbreviation “UAG” are intended to mean peptides that have the amino acid sequence specified in SEQ ID NO: 1 which amino acid sequence is:

(SEQ ID NO: 1)
Gly-Ser-Ser-Phe-Leu-Ser-Pro-Glu-His-Gln-Arg-Val-
Gln-Gln-Arg-Lys-Glu-Ser-Lys-Lys-Pro-Pro-Ala-Lys-
Leu-Gln-Pro-Arg

Unacylated ghrelin may also be referred to as UAG (1-28).

Naturally-occurring variations of UAG include peptides that contain substitutions, additions or deletions of one or more amino acids which result due to discrete changes in the nucleotide sequence of the encoding ghrelin gene or alleles thereof or due to alternative splicing of the transcribed RNA. It is understood that the changes do not substantially affect the properties, pharmacological and biological characteristics of unacylated ghrelin variants. Those peptides may be in the form of salts. Particularly the acidic functions of the molecule may be replaced by a salt derivative thereof such as, but not limited to, a trifluoroacetate or an acetate salt.

By “peptide”, “polypeptide” or “protein” is meant any chain of amino acids, regardless of length or post-translational modification (e.g., glycosylation or phosphorylation), or chemical modification, or those containing unnatural or unusual amino acids such as D-Tyr, ornithine, amino-adipic acid. The terms are used interchangeably in the present application.

The expressions “fragments” and “fragments thereof” refer to amino acid fragments of a peptide such as UAG. Fragments of UAG are shorter than the amino acid sequence depicted in SEQ ID NO: 1, therefore are shorter than 28 amino acid residues. Fragments of UAG may be 27, 26, 25, 24, 23, 22, 21, 20, 19, 18, 17, 16, 15, 14, 13, 12, 11, 10, 9, 8, 7, 6, 5 or 4 amino acid residues in length. For example, fragments of UAG may have the amino acid sequences depicted in Table 1 below:

TABLE 1
UAG fragments

| | SEQ ID | |
|---|---|---|
| Fragment | NO: | Amino Acid Sequence |
| UAG (1-14) | 2 | Gly-Ser-Ser-Phe-Leu-Ser-Pro-Glu-His-Gln-Arg-Val- |
| | | Gln-Gln |
| UAG (1-18) | 3 | Gly-Ser-Ser-Phe-Leu-Ser-Pro-Glu-His-Gln-Arg-Val- |
| | | Gln-Gln-Arg-Lys-Glu-Ser |
| UAG (1-5) | 4 | Gly-Ser-Ser-Phe-Leu |
| UAG (17-28) | 5 | Glu-Ser-Lys-Lys-Pro-Pro-Ala-Lys-Leu-Gln-Pro-Arg |
| UAG (6-13) | 6 | Ser-Pro-Glu-His-Gln-Arg-Val-Gln |
| UAG (8-13) | 7 | Glu-His-Gln-Arg-Val-Gln |
| UAG (8-12) | 8 | Glu-His-Gln-Arg-Val |
| UAG (6-18) | 9 | Ser-Pro-Glu-His-Gln-Arg-Val-Gln-Gln-Arg-Lys-Glu-Ser |
| UAG (8-11) | 10 | Glu-His-Gln-Arg |
| UAG (9-12) | 11 | His-Gln-Arg-Val |
| UAG (9-11) | 29 | His-Gln-Arg |
| UAG (14-1) | 30 | Gln Gln Val Arg Gln His Glu Pro Ser Leu Phe Ser Ser Gly |

Any other fragments of UAG that preserve the biological activity of UAG are encompassed by the present invention. Some UAG fragments have been reported in U.S. Pat. No. 8,222,217; U.S. Pat. No. 8,318,664; U.S. Pat. No. 8,476,408 and in U.S. Patent Applications 2010/0016226 and WO 2009/150214, which are all incorporated herein in their entirety by reference, wherein it has been demonstrated that the smallest UAG fragment to retain the biological activity of UAG is UAG (9-12) depicted herein as SEQ ID NO: 11.

For simplicity, UAG, UAG fragments and UAG analogs are collectively referred to herein as “the peptides as defined herein” or as “the peptides useful in the present invention” or as “the peptide of the invention”.

In one embodiment of the present invention, peptides such as UAG, fragments or analogs thereof, are used in a form that is “purified”, “isolated” or “substantially pure”. The peptides are “purified”, “isolated” or “substantially pure” when they are separated from the components that naturally accompany them. Typically, a compound is substantially pure when it is at least 60%, 65%, 70%, 75%, 80%, 85%, 90%, 91%, 92%, 93%, 94%, 95%, 96%, 97%, 98%, or 99%, by weight, of the total material in a sample.

The expressions “biological activity” or “biological property”, or the term “activity” in reference to the peptides as defined herein, are used interchangeably herein and refer to the biological, cellular and/or pharmaceutical abilities of the peptides as defined herein and include, but are not limited to, the capacity of replacing UAG in the biological functions of UAG as described in U.S. Pat. Nos. 7,485,620; 7,666,833; 8,071,368; 7,825,090; 8,222,217; 8,318,666 and 8,476,408 and in U.S. Patent Applications 2010/0016226 and 2013/0157936 or as described in the present application, such as, but not limited to, in protecting against oxidative stress-induced damage, reducing oxidative stress-induced damage, protecting against cell injuries induced by oxidative stress, protecting against ROS-induced cell injuries, inducing muscle regeneration, reducing functional impairment of muscle cells, inducing skeletal muscle regeneration, having antioxidant effect on oxidative-damaged cells, reducing functional impairment of skeletal muscle cells, protecting satellite cells from oxidative stress-induced damage and modulating cellular levels of SOD-2.

Some analogs of UAG have been reported in U.S. Pat. No. 8,222,217; U.S. Pat. No. 8,318,664; U.S. Pat. No. 8,476,408 and in U.S. Patent Applications 2010/0016226 and WO 2009/150214, all incorporated herein in their entirety by reference. Simple structural analogs comprise peptides showing homology with UAG as set forth in SEQ ID NO: 1 or homology with any fragment thereof. An example of an analog of AG is an isoform of Ghrelin-28, des Gln-14 Ghrelin (a 27 amino acid peptide possessing serine 3 modification by n-octanoic acid) which is shown to be present in stomach. It is functionally identical to AG in that it binds to GHSR-1a with similar binding affinity, elicits Ca²⁺ fluxes in cloned cells and induces GH secretion with similar potency as Ghrelin-28. It is expected that UAG also has a des Gln-14 UAG that is functionally identical to UAG.

The expressions “analog of unacylated ghrelin”, “analog of fragments of unacylated ghrelin” and “analogs thereof” refer to both structural and functional analogs of UAG or fragments thereof which are, inter alia, capable of replacing UAG in protecting against oxidative stress-induced damage, reducing oxidative stress-induced damage, protecting against cell injuries induced by oxidative stress, protecting against ROS-induced cell injuries, inducing muscle regeneration, reducing functional impairment of muscle cells, inducing skeletal muscle regeneration, having antioxidant effect on oxidative-damaged cells, reducing functional impairment of skeletal muscle cells, protecting satellite cells from oxidative stress-induced damage and modulating cellular levels of SOD-2.

Preferred analogs of UAG and preferred analogs of fragments of UAG are those that vary from the native UAG sequence or from the native UAG fragment sequence by conservative amino acid substitutions, those that substitute a residue with another of like characteristics. Typical substitutions include those among Ala, Val, Leu and Ile; among Ser and Thr; among the acidic residues Asp and Glu; among Asn and Gln; among the basic residues Lys and Arg; and among the aromatic residues Phe and Tyr. Particularly preferred are analogs in which several, for example, but not limited to, 5-10, 1-5, or 1-2 amino acids are substituted, deleted, or added in any combination. For example, the analogs of UAG may differ in sequence from UAG by 1, 2, 3, 4, 5, 6, 7, 8, 9 or 10 amino acid substitutions (preferably conservative substitutions), deletions, or additions, or combinations thereof.

There are provided herein, analogs of the peptides as defined herein that have at least 50%, 55%, 60%, 65%, 70%, 75%, 80%, 85%, 90%, 91%, 92%, 93%, 94%, 95%, 96%, 97%, 98% or 99% sequence homology or sequence identity with the amino acid sequences described herein over its full length, and sharing at least one of the metabolic effects or biological activity of UAG. A person skilled in the art would readily identify an analog sequence of unacylated ghrelin or an analog sequence of a fragment of unacylated ghrelin. Examples of analogs of UAG are provided in Table 2 below:

TABLE 2
UAG analogs

| | SEQ ID | |
|---|---|---|
| Analog | NO: | Amino acid sequence |
| (Asp)8 UAG (6-13)NH₂ | 12 | Ser-Pro-Asp-His-Gln-Arg-Val-Gln |
| (Lys)11 UAG (6-13)NH₂ | 13 | Ser-Pro-Glu-His-Gln-Lys-Val-Gln |
| (Gly)6 UAG (6-13)NH₂ | 14 | Gly-Pro-Glu-His-Gln-Arg-Val-Gln |
| (Ala)6 UAG (6-13)NH₂ | 15 | Ala-Pro-Glu-His-Gln-Arg-Val-Gln |
| (Ala)7 UAG (6-13)NH₂ | 16 | Ser-Ala-Glu-His-Gln-Arg-Val-Gln |
| (Ala)8 UAG (6-13)NH₂ | 17 | Ser-Pro-Ala-His-Gln-Arg-Val-Gln |
| (Ala)9 UAG (6-13)NH₂ | 18 | Ser-Pro-Glu-Ala-Gln-Arg-Val-Gln |
| (Ala)10 UAG (6-13)NH₂ | 19 | Ser-Pro-Glu-His-Ala-Arg-Val-Gln |
| (Ala)11 UAG (6-13)NH₂ | 20 | Ser-Pro-Glu-His-Gln-Ala-Val-Gln |
| (Ala)12 UAG (6-13)NH₂ | 21 | Ser-Pro-Glu-His-Gln-Arg-Ala-Gln |
| (Ala)13 UAG (6-13)NH₂ | 22 | Ser-Pro-Glu-His-Gln-Arg-Val-Ala |
| (Acetyl-Ser)6 UAG (6-13)NH₂ | 23 | Ac-Ser-Pro-Glu-His-Gln-Arg-Val-Gln |
| (Acetyl-Ser)6, (DPro)7 UAG (6-13)NH₂ | 24 | Ac-Ser-Pro-Glu-His-Gln-Arg-Val-Gln |
| Cyclo (6-13) UAG (also referred to as | 25 | Ser-Pro-Glu-His-Gln-Arg-Val-Gln (cycl) |
| cyclic UAG (6-13) or UAG (6-13) cyclic) | | |
| Cyclo (8,11), Lys 11, UAG (6-13)amide | 26 | Ser-Pro-Glu-His-Gln-Lys-Val-Gln-amide |
| Cyclo (8,11), Acetyl-Ser6, Lys 11, | 27 | Ac-Ser-Pro-Glu-His-Gln-Lys-Val-Gln (cycl) |
| UAG (6-13)-amide | | |
| Acetyl-Ser6, Lys 11, UAG (6-13)NH₂ | 28 | Ac-Ser-Pro-Glu-His-Gln-Lys-Val-Gln-NH₂ |

Analogs of UAG or analogs of fragments thereof are, for example, analogs obtained by alanine scans, by substitution with D-amino acids or with synthetic amino acids or by cyclization of the peptide. Analogs of UAG or fragments thereof may comprise a non-naturally encoded amino acid, wherein the non-naturally encoding amino acid refers to an amino acid that is not one of the common amino acids or pyrrolysine or selenocysteine, or an amino acid that occur by modification (e.g. post-translational modification) of naturally encoded amino acid (including, but not limited to, the 20 common amino acids or pyrrolysine and selenocysteine) but are not themselves incorporated into a growing polypeptide chain by the translation complex. Examples of such non-naturally-occurring amino acids include, but are not limited to, N-acetylglucosaminyl-L-serine, N-acetylglucosaminyl-L-threonine and O-phosphotyrosine.

As used herein, the term “modified” refers to any changes made to a given peptide, such as changes to the length of the peptide, the amino acid sequence, chemical structure, co-translational modification, or post-translational modification of a peptide.

The term “post-translational modification” refers to any modification of a natural or non-natural amino acid that occurs to such an amino acid after it has been incorporated into a peptide chain. The term encompasses, by way of example only, co-translational in vivo modifications, co-translational in vitro modifications (such as in cell-free translation system), post-translational in vivo modifications, and post-translational in vitro modifications. Examples of post-translational modifications are, but are not limited to, glycosylation, acetylation, acylation, amidation, carboxylation, phosphorylation, PEGylation, addition of salts, amides or esters, in particular C-terminal esters, and N-acyl derivatives of the peptides as defined herein. The types of post-translational modifications are well known.

Certain peptides according to the present invention may also be in cyclic form, such that the N- or C-termini are linked head-to-tail either directly, or through the insertion of a linker moiety, such moiety itself generally comprises one or more amino acid residues as required to join the backbone in such a manner as to avoid altering the three-dimensional structure of the peptide with respect to the non-cyclic form. Such peptide derivatives may have improved stability and bioavailability relative to the non-cyclic peptides. Examples of cyclic peptides of the present invention include: cyclic UAG (1-14), cyclic UAG (1-18), cyclic UAG (17-28), cyclic UAG (6-13), cyclic UAG (8-13), cyclic UAG (8-12), cyclic UAG (8-11), cyclic UAG (9-12) and cyclic UAG (9-11) as well as the peptides identified in Table 2.

Methods for cyclizing peptides are well known in the art and for example may be accomplished by disulfide bond formation between two side chain functional groups, amide or ester bond formation between one side chain functional group and the backbone α-amino or carboxyl function, amide or ester bond formation between two side chain functional groups, or amide bond formation between the backbone α-amino and carboxyl functions. These cyclization reactions have been traditionally carried out at high dilution in solution. Cyclization is commonly accomplished while the peptide is attached to the resin. One of the most common ways of synthesizing cyclic peptides on a solid support is by attaching the side chain of an amino acid to the resin. Using appropriate protection strategies, the C-and N-termini can be selectively deprotected and cyclized on the resin after chain assembly. This strategy is widely used, and is compatible with either tert-butyloxycarbonyl (Boc) or 9-fluorenylmethoxycarbonyl (Fmoc) protocols. However, it is restricted to peptides that contain appropriate side chain functionality to attach to the solid support. A number of approaches may be used to achieve efficient synthesis of cyclic peptides. One procedure for synthesizing cyclic peptides is based on cyclization with simultaneous cleavage from the resin. After an appropriate peptide sequence is assembled by solid phase synthesis on the resin or a linear sequence is appended to resin, the deprotected amino group can react with its anchoring active linkage to produce protected cyclic peptides. In general, a final deprotection step is required to yield the target cyclic peptide.

Lactamazation, a form of cyclization, may be performed to form a lactam bridge using Fmoc synthesis, amino acids with different protecting groups at the lateral chains may be introduced, such as, but not limited to, aspartic acid (or glutamic) protected with allyl ester at the beta ester (or gamma ester for glutamic acid) and lysine protected with allyloxy carbamate at the N-ε. At the end of the synthesis, with the N-terminus of the peptide protected with Fmoc, Boc or other protecting group different from Alloc, the allyl and alloc protecting groups of aspartic acid and lysine may be deprotected with, for example, palladium (0) followed by cyclization using PyAOP (7-Azabenzotriazol-1-yloxytris(pyrrolidino) phosphonium-hexafluorophosphate) to produce the lactam bridge.

Unless otherwise indicated, an amino acid named herein refers to the L-form. Well recognized abbreviations in the art will be used to describe amino acids, including levorotary amino acids (L-amino acids or L or L-form) and dextrorotatory amino acids (D-amino acids or D or D-form), Alanine (Ala or A), Arginine (Arg or R), Asparagine (Asn or N), Aspartic acid (Asp or D), Cysteine (Cys or C), Glutamic acid (Glu or E), Glutamine (Gln or Q), Glycine (Gly or G), Histidine (His or H), Isoleucine (Ile or I), Leucine (Leu or L), Lysine (Lys or K), Methionine (Met or M), Phenylalanine (Phe or F), Proline (Pro or P), Serine (Ser or S), Threonine (Thr or T), Tryptophan (Trp or W), Tyrosine (Tyr or Y) and Valine (Val or V). An L-amino acid residue within the native peptide sequence may be altered to any one of the 20 L-amino acids commonly found in proteins or any one of the corresponding D-amino acids, rare amino acids, such as, but not limited to, 4-hydroxyproline or hydroxylysine, or a non-protein amino acid, such as P-alanine or homoserine.

UAG peptides or fragments or analogs thereof may also be part of a fusion protein. It is often advantageous to include an additional amino acid sequence such as a signal sequence or a leader sequence which contains for example secretory sequences, pro-sequences, linker sequences. Some of these additional sequences may aid in purification such as multiple histidine residues (HA-tag) or an additional sequence for stability during recombinant production. Some of these additional sequences may aid in directing the peptides as defined herein to a specific target in an organism such as in targeting the peptides as defined herein to a specific organ or tissue or targeting the peptides as defined herein to a specific organelle within a cell.

In some implementations, UAG or fragments or analogs thereof may be in a protein precursor format (i.e., pro-UAG, pro-UAG fragment, pro-AUG analog, pre-pro-UAG, pre-pro-UAG fragment or pre-pro-UAG analog). In some implementations, a leader sequence may be attached to target the peptides as defined herein to the mitochondrial. In this implementation, the leader sequence is a mitochondrial leader sequence. Mitochondrial leader sequences are well known in the art.

The additional amino acids or sequence may be linked to at the N-terminal or at the C-terminal of the peptide or may be linked to any amino acid of the sequences located between the N- and the C-terminal to give rise the UAG peptides or fragments or analogs thereof having a linker moiety.

Any other analogs of UAG or fragments thereof or any other modified UAG or fragments thereof that preserve the biological activity of the full length UAG are encompassed by the present invention.

As used herein, the term “homology” refers to sequence similarity between two peptides while retaining an equivalent biological activity. Homology can be determined by comparing each position in the aligned sequences. A degree of homology between amino acid sequences is a function of the number of identical or matching amino acids at positions shared by the sequences so that a “homologous sequence” refers to a sequence sharing homology and an equivalent function or biological activity. Assessment of percent homology is known by those of skill in the art.

Methods to determine homology, identity and similarity of peptides are codified in publicly available computer programs. Preferred computer program methods to determine identity and similarity between two sequences include, but are not limited to, the GCG program package, BLASTP, BLASTN, and FASTA. The BLAST X program is publicly available from NCBI and other sources. The well known Smith Waterman algorithm may also be used to determine identity.

Preferred parameters for peptide sequence comparison include the following: Algorithm: Needleman and Wunsch, J. Mol. Biol. 48: 443-453 (1970); Comparison matrix: BLOSSUM62 from Hentikoff and Hentikoff, Proc. Natl. Acad. Sci. USA. 89:10915-10919 (1992); Gap Penalty: 12; Gap Length Penalty: 4. A program useful with these parameters is publicly available as the “gap” program from Genetics Computer Group, Madison, Wis. The aforementioned parameters are the default parameters for amino acid sequence comparisons (along with no penalty for end gaps).

The peptides useful in the methods of the present invention may be chemically synthesized by any of the methods well known in the art. Suitable methods for synthesizing the protein include, for example those described by Stuart and Young in “Solid Phase Peptide Synthesis” Second Edition, Pierce Chemical Company (1984), and in “Solid Phase Peptide Synthesis” Methods Enzymol. 289, Academic Press, Inc, New York (1997). General methods and synthetic strategies used in providing functional and structural analogs of UAG or fragments thereof are commonly used and well known in the art and are described in publications such as: “Peptide synthesis protocols” ed, M. W. Pennigton & B. M. Dunn. Methods in Molecular Biology. Vol 35. Humana Press, NJ., 1994; “Solid phase peptide synthesis” by Stewart and Young, W. h Freeman & Co., San Francisco, 1969 and Erickson and Merrifield; and “The Proteins” Vol. 2, p. 255 et seq. (Ed. Neurath and Hill), Academic Press, New York, 1976.

The peptides as defined herein may be prepared in any suitable methods as known in the art. Such peptides include isolated naturally occurring peptides, recombinantly produced peptides, synthetically produced peptides, or peptides produced by a combination of these methods. Means and methods for preparing such peptides are well known in the art.

Certain aspects of the invention use UAG polynucleotides. These include isolated polynucleotides which encode the UAG polypeptides, fragments and analogs defined in the application. As used herein, the term “polynucleotide” refers to a molecule comprised of a plurality of deoxyribonucleotides or nucleoside subunits. The linkage between the nucleoside subunits can be provided by phosphates, phosphonates, phosphoramidates, phosphorothioates, or the like, or by nonphosphate groups as are known in the art, such as peptoid-type linkages utilized in peptide nucleic acids (PNAs). The linking groups can be chiral or achiral. The oligonucleotides or polynucleotides can range in length from 2 nucleoside subunits to hundreds or thousands of nucleoside subunits. While oligonucleotides are preferably 5 to 100 subunits in length, and more preferably, 5 to 60 subunits in length, the length of polynucleotides can be much greater (e.g., up to 100). The polynucleotide may be any of DNA and RNA. The DNA may be in any form of genomic DNA, a genomic DNA library, cDNA derived from a cell or tissue, and synthetic DNA. Moreover, the present invention may, in certain aspects, use vectors which include bacteriophage, plasmid, cosmid, or phagemid.

### B) Method of Use, Therapeutic Methods and Compositions

In one embodiment, the peptides of the present invention may be useful in protecting against oxidative stress-induced damage, more particularly against oxidative stress-induced tissue damage. In one implementation of this embodiment, the peptides as defined herein may be useful in protecting a subject against oxidative stress-induced damage. Subjects in need of protection against oxidative stress-induced damage include those subjects who are suffering from a disease or a condition associated with oxidative stress such as, but not limited to, neurodegenerative diseases (such as, but not limited to, Parkinson's disease, Lou Gehrig's disease, Alzheimer's disease and Huntington's disease), atherosclerosis, heart failure, myocardial infarction, ischemia, tissue injury following ischemia-reperfusion, reperfusion injury following organ transplantation, stroke, coronary heart disease, peripheal arterial disease, injury associated with cardiopulmonary bypass surgery, fragile X syndrome, sickle cell disease, lichen planus, vitiligo, autism, chronic fatigue syndrome, preeclampsia, diabetes, non-alcoholic fatty liver disease (NAFLD), metabolic syndrome, mitochondrial encephalopathies, Wilson's disease, myotonic dystrophy type I and symptoms of and conditions associated with aging such as macular degeneration and wrinkles.

A subject in need thereof can be any mammal, including, for example, farm animals, such as sheep, pigs, cows, and horses; pet animals, such as dogs and cats; laboratory animals, such as rats, mice and rabbits. In a preferred implementation, the subject is a human.

As used herein, the expression “oxidative stress” refers to an imbalance between the systemic manifestation of reactive oxygen species and a biological system's ability to readily detoxify the reactive intermediates or to repair the resulting damage. Oxidative stress may be caused by abiotic or environmental stress conditions including metal toxicity, temperature stress, osmotic stress, drought stress or salt stress.

As used herein, the expression “oxidative damage” refers to damage that is caused by free radicals, such as reactive oxygen species (ROS) and/or reactive nitrogen species (RNS). Examples of such radicals include, but are not limited to, hydroxyl radical (HO⁻), superoxide anion radical (O₂⁻), nitric oxide (NO), hydrogen peroxide (H₂O₂), hypochlorous acid (HOCl) and peroxynitrite anion (ONOO⁻).

As used herein, the expression “oxidative stress-induced damage” refers to damage such as, but not limited to, damage to a tissue and/or an organ of a subject which is isolated or not from the subject and that is induced or caused by oxidative stress.

The expression “diseases or conditions associated with oxidative stress-induced damage” or “oxidative stress-associated diseases or conditions”, as used herein, refers to a disease, a medical disorder or a medical condition (including syndromes) wherein the onset or progression thereof is promoted by oxidative stress, in particular wherein the healthy function of one or more organelles, non-organelle subcellular structures, cell, cell types, tissues, tissue types, organs, or organ systems, particularly the mitochondria, is impaired by the action of oxidizing agents, particularly ROS. The action of oxidizing agents need not be the only route by which impairment of healthy function occurs in the course of a disease for the disease to be an oxidative stress-induced disease. In some implementations, the oxidative stress-induced damage-associated diseases or conditions are a mitochondrial dysfunction related disease or condition. Mitochondrial dysfunction relates to abnormalities in mitochondria and diseases and conditions associated with or involving decreased mitochondrial function.

As used herein, the expression “protecting against oxidative stress-induced damage” includes preventing the generation of free radicals and hydrogen peroxide directly and/or enhancing the capacity to trap the free radicals and the hydrogen peroxide.

Oxidative stress-induced damage can occur in an organ, a tissue or in a cell/population of cells of the subject. Examples of organs which can be affected by oxidative stress-induced damage include, but are not limited to, brain, heart, kidneys, liver and lungs. Examples of tissues that can be affected by oxidative stress-induced damage include, but are not limited to connective tissues, muscle tissues, nervous tissues, epithelial tissues and endothelial tissues. Examples of muscle tissues include smooth muscle tissues, skeletal muscle tissues and cardiac muscle tissues. Examples of cells that can be affected by oxidative stress-induced damage include, but are not limited to, endothelial cells, muscle cells, cardiomyocytes, epithelial cells, nervous system cells and cells of the tissues and organs discussed above.

A subject in need thereof may also be a subject undergoing a treatment associated with oxidative stress-induced damage. For example, the subject may be undergoing reperfusion. As used herein, the term “reperfusion” refers to the restoration of blood flow to any organ or tissue in which the flow of blood is decreased or blocked. The restoration of blood flow during reperfusion leads to respiratory burst and formation of free radicals. Decreased or blocked blood flow may be due for example, to hypoxia or ischemia. The loss or severe reduction in blood supply during hypoxia or ischemia may, for example, be due to thromboembolic stroke, coronary atherosclerosis, or peripheral vascular disease. For instance, cardiac muscle ischemia or hypoxia is commonly caused by atherosclerotic or thrombotic blockages which lead to the reduction or loss of oxygen delivery to the cardiac tissues by the cardiac arterial and capillary blood supply. Such cardiac ischemia or hypoxia may cause pain and necrosis of the affected cardiac muscle, and ultimately may lead to heart failure. Ischemia or hypoxia in skeletal muscle or smooth muscle may arise from similar causes. For example, ischemia or hypoxia in intestinal smooth muscle or skeletal muscle of the limbs may also be caused by atherosclerotic or thrombotic blockages.

Liver damage caused by a toxic agent is another condition which is associated with an inflammatory process and oxidative stress. The toxic or infectious agent can be any agent which causes damage to the liver. For example, the toxic agent can cause apoptosis and/or necrosis of liver cells. Examples of such agents include alcohol, and medication, such as prescription and non-prescription drugs taken to treat a disease or condition.

Oxidative stress-induced damage may also be caused by lipid peroxidation. Lipid peroxidation refers to oxidative modification of lipids. The lipids can be present in the membrane of a cell. This modification of membrane lipids typically results in change and/or damage to the membrane function of a cell. In addition, lipid peroxidation can also occur in lipids or lipoproteins exogenous of a cell. For example, low-density lipoproteins are susceptible to lipid peroxidation. An example of a condition associated with lipid peroxidation is atherosclerosis.

In an implementation of this embodiment, the peptides as defined herein may be used for reducing oxidative stress-induced damage in a subject. Oxidative stress-induced damage is considered to be “reduced” if the amount of oxidative stress-induced damage in a subject, an organ, a tissue or in a cell or in a population of cells is decreased after administration of an effective amount of UAG, fragments and analogs thereof. Typically, the oxidative stress-induced damage is considered to be reduced if the oxidative stress-induced damage is decreased by at least about 5%, preferably at least about 10%, more preferably at least about 25%, more preferably at least about 50%, even more preferably at least about 75%, and most preferably at least about 90%.

In another embodiment, the peptides useful in the present invention may also be used for protecting a tissue and/or an organ against oxidative stress-induced damage.

In one implementation of this embodiment, the peptides useful in the present invention may be used for protecting a tissue and/or an organ from oxidative stress-induced damage prior to or after transplantation. For example, a removed tissue or organ, when subjected to reperfusion after transplantation can be susceptible to oxidative stress-induced damage. Therefore, the peptides as defined herein may be used to reduce oxidative damage from reperfusion of the transplanted tissue or organ. The removed tissue or organ may be any tissue or organ suitable for transplantation and/or engraftment and once treated with the peptides as defined herein may be transplanted in a subject as a graft. Examples of such tissues or organs include the heart, liver, kidneys, lung and pancreatic islets. The removed tissue or organ is placed in a suitable medium, such as in a standard buffered solution commonly used in the art. The methods and techniques for transplantation are also well known in the art.

In yet another embodiment, the peptides as defined herein may be used for protecting a cell or a population of cells against oxidative stress-induced damage. A cell or a population of cells in need of such protection is generally a cell in which the cell membrane or DNA of the cell has been damaged by free radicals (e.g., ROS) or in which the mitochondria is dysfunctional. Examples of such cells include, but are not limited to, pancreatic islet cells, myocytes, endothelial cells, neuronal cells, stem cells, etc. The cells can be tissue cultured cells. Alternatively, the cells may be obtained from a subject. In one instance, the cells can be damaged by oxidative stress as a result of an insult. Such insults include, for example, a disease or condition (e.g., diabetes, etc.). For example, pancreatic islet cells damaged by oxidative stress as a result of diabetes can be obtained from a subject suffering from diabetes.

In one embodiment, the peptides useful in the present invention may be used for preventing and/or treating oxidative stress-induced damage in a subject, a tissue, an organ, a cell or a population of cells in need thereof.

In a further embodiment, the peptides useful in the present invention may be used for protecting a muscle tissue against oxidative stress-induced damage. In one implementation of this embodiment, the peptides as defined herein may be used to promote regeneration of a muscle tissue. In other implementation of this embodiment, the peptides as defined herein may be used to improve tissue regeneration and functional recovery of muscle under oxidative stress conditions. Examples of muscle tissue in need of protection against oxidative stress include, but are not limited to, an ischemia-induced damaged muscle. In any of these implementations, the muscle tissue is a skeletal muscle tissue, a smooth muscle tissue or a cardiac muscle tissue. In one specific but non-limiting example, the muscle tissue is an ischemia-induced damaged skeletal muscle.

In a further embodiment, the peptides defined herein may be used for reducing oxidative stress conditions. The phrase “oxidative stress conditions” as used herein, refers to conditions that results in oxidative stress and elevate the ROS level beyond the normal level, resulting in e.g. destruction of cells and cellular components (e.g., mitochondria), causing cells to lose their structure and/or function, and/or cell death. Particular oxidative stress conditions are those that result in or are related with mitochondrial dysfunction.

In a further embodiment, the peptides as defined herein may be used to protect a tissue and/or an organ from oxidative stress-induced damage in which metabolic intermediates have accumulated. As used herein, the expression “metabolic intermediates” refers to molecules which are the precursors or metabolites of biologically significant molecules and wherein the accumulation of which may create an oxidative stress and lead to damage.

In a further embodiment, the peptides as defined herein may be used to protect a tissue and/or an organ against oxidative damage wherein which tissue and/or an organ the oxygen supply has been interrupted. Oxygen interruption may be caused by, for example, an ischemic injury which itself may be the result of a myocardial infarction, stroke, and other thrombolytic events.

In yet a further embodiment, the peptides as defined herein may be used to protect a tissue, an organ and/or a population of cells from oxidative stress-induced damage wherein the cells of the tissue, organ and/or the population of cells have a defective aerobic metabolism. As used herein, the expression “aerobic metabolism” refers to the creation of energy through the combustion of carbohydrates and fats in the presence of oxygen.

In yet a further embodiment, the peptides as defined herein may be used to protect a tissue, an organ and/or a population of cells from oxidative stress-induced damage wherein the cells of the tissue, organ and/or the population of cells have defective mitochondrial electron transfer chain and increased ROS generation.

In yet a further embodiment, the peptides as defined herein may be used to improve repair of a tissue that has been or that is under oxidative stress. As used herein, the expression “tissue repair” refers to restoring the tissue to a sound condition after it has been damaged or injured. In some implementations of this embodiment, the tissue in need of repair is a muscle tissue (such as skeletal muscle tissue), a smooth muscle tissue or a cardiac muscle tissue.

In still a further embodiment, the peptides as defined herein may be used to improve or ameliorate an oxidative stress-induced damage-associated disease or condition, or to improve or ameliorate oxidative stress resistance of a tissue, organ, a cell or a population of cells. The term “ameliorating” refers to improving the condition of a subject suffering or at risk of suffering from the disease or condition. Ameliorating can comprise one or more of the following: a reduction in the severity of a symptom of the disease or condition, a reduction in the extent of a symptom of the disease or condition, a reduction in the number of symptoms of the disease or condition, a reduction in the number of disease agents, a reduction in the spread of a symptom of the disease or condition, a delay in the onset of a symptom of the disease or condition, a delay in disease onset or condition onset, or a reduction in the time between onset of the disease or condition and remission of the disease or condition.

In still a further embodiment, the peptides as defined herein may be used to improve or ameliorate oxidative stress resistance of a tissue, organ, a cell or a population of cells.

In still a further embodiment, the peptides as defined herein may be used to increase oxidative stress tolerance in an organ, tissue and/or cell. As used herein, the expression “increased oxidative stress tolerance” comprises, increasing tolerance in an organ, tissue and/or cell to oxidative stress conditions, whether the organ, tissue or cell already has some degree of tolerance to the oxidative stress, or whether the organ, tissue or cell is being provided with tolerance to that oxidative stress, anew.

The terms “resistance” and “tolerance” as used herein, encompass protection against oxidative stress ranging from a delay to substantially a complete inhibition of alteration in cellular metabolism, reduced cell growth and/or cell death caused by stress conditions, particularly oxidative stress conditions.

In another embodiment, the peptides as defined herein may be used to improve muscle tissue regeneration in a subject. As used herein, the expression “muscle tissue regeneration” refers to the process by which new muscle fibers form from muscle progenitor cells such as SCs. The useful improvement for regeneration confers an increase in the number of new fibers by at least 1%, more preferably by at least 10%, more preferably by at least 15%, more preferably by at least 20%, more preferably by at least 25% and most preferably by at least 50%. The muscle tissue in need of regeneration may be a cardiac muscle tissue, a smooth muscle tissue or a skeletal muscle tissue. In one implementation of this embodiment, the muscle tissue in need of regeneration is a skeletal muscle tissue. In another implementation of this embodiment, the skeletal muscle tissue in need of regeneration is an ischemic skeletal muscle tissue. In a further implementation of this embodiment, the skeletal muscle tissue in need of regeneration is an ischemia-reperfused skeletal muscle tissue.

In some implementations of these embodiments, the methods of the present invention include the step of administering an effective amount of UAG or of a fragment or an analog thereof as defined herein which shares the same potential therapeutic indication as UAG itself to the subject in need of such administration. The peptides as defined herein are administered to a subject in an amount effective in protecting from oxidative stress-induced damage. The effective amount is determined during pre-clinical trials and clinical trials by methods known in the art.

In some implementations of these embodiments, the methods of the present invention include the step of contacting the tissues, organs or population of cells with an effective amount of UAG or of a fragment or an analog thereof as defined herein which shares the same potential therapeutic indication as UAG itself. The peptides useful in the present invention are put in contact with the tissues, organs or population of cells in an amount effective in protecting from oxidative stress-induced damage.

Such peptides comprise the amino acid sequence set forth in SEQ ID NO: 1, or comprises any fragment or any analog thereof such as for example, those described in the above tables. The actions of UAG have previously been shown to be conserved by fragments UAG (6-13) (SEQ ID NO: 6), UAG (8-13) (SEQ ID NO: 7), UAG (8-12) (SEQ ID NO: 8), UAG (8-11) (SEQ ID NO: 12), UAG (9-12) (SEQ ID NO: 11) and UAG (9-11) (SEQ ID NO: 29). U.S. Pat. Nos. 8,222,217 and 8,318,664, incorporated herein in their entirety, have shown that these fragments retain the activity of UAG full length on glucose, insulin and lipid metabolisms. A peptide with the inverse sequence of UAG (1-14) (SEQ ID NO: 3) and named UAG (14-1) (SEQ ID NO: 30) was used as a negative control in the experiments testing UAG fragments. UAG (8-11) (SEQ ID NO: 10) was shown to be the smallest UAG fragment to retain UAG activities. The results provided herein further indicate that UAG fragments, such as for example, UAG (6-13) (SEQ ID NO: 6) and cyclic UAG (6-13) (SEQ ID NO: 25) retain the functions of UAG.

As used herein, the term “treatment” refers to both therapeutic treatments as well as to prophylactic measures. Those in need of treatment include those already with the disorder, disease or condition as well as those in which the disease, disorder or condition is to be prevented. Those in need of treatment are also those in which the disorder, disease or condition has occurred and left after-effects or scars. Treatment also refers to administering a therapeutic substance effective to improve or ameliorate, diminish symptoms associated with a disease, a disorder or a condition to lessen the severity of or cure the disease, disorder or condition, or to prevent the disease, disorder or condition from occurring or reoccurring.

It is a further embodiment, the present invention provides for a pharmaceutical composition incorporating at least one of the peptides as defined herein.

For therapeutic and/or pharmaceutical uses, the peptides as defined herein may be formulated for, but not limited to, intravenous, subcutaneous, transdermal, topical, oral, buccal, sublingual, nasal, inhalation, pulmonary, or parenteral administration according to conventional methods. Intravenous injection may be by bolus or infusion over a conventional period of time. The peptides as defined herein may also be administered directly to a target site within a subject e.g., by biolistic delivery to an internal or external target site or by catheter to a site in an artery. The peptides can be injected directly into coronary artery during, for example, angioplasty or coronary bypass surgery, or applied onto coronary stents. Other routes of administration include intracerebroventricularly or intrathecally.

In one implementation of this embodiment, the peptides as defined herein are administered as a bolus. Accordingly, the medicament is administered as a bolus prior to meal, wherein the bolus comprises an effective amount of UAG, a fragment and/or an analog thereof of a salt thereof. The bolus may be administered one, twice, three times or more daily or may be administered according to other dosage regimens.

Suitable dosage regiments are determined taking into account factors well known in the art such as, but not limited to, type of subject being dosed, the age, the weight, the sex and the medical condition of the subject, the route of administration, the desired affect, etc.

Active ingredients, such as the peptides as defined herein, may be administered orally as a suspension and can be prepared according to techniques well known in the art of pharmaceutical formulation and may contain, but not be limited to, microcrystalline cellulose for imparting bulk, alginic acid or sodium alginate as a suspending agent, methylcellulose as a viscosity enhancer, and sweeteners/flavoring agents. As immediate release tablets, these compositions may contain, but are not limited to microcrystalline cellulose, dicalcium phosphate, starch, magnesium stearate and lactose and/or other excipients, binders, extenders, disintegrants, diluents and lubricants. The active ingredients may be administered by way of a controlled-release delivery system.

Administered by nasal aerosol or inhalation formulations may be prepared, for example, as solutions in saline, employing benzyl alcohol or other suitable preservatives, absorption promoters to enhance bioavailability, employing fluorocarbons, and/or employing other solubilizing or dispersing agents.

The peptides as defined herein may be administered in intravenous (both bolus and infusion), intraperitoneal, subcutaneous, topical with or without occlusion, or intramuscular form. When administered by injection, the injectable solution or suspension may be formulated using suitable non-toxic, parenteral-acceptable diluents or solvents, well known in the art.

The peptides as defined herein may also be formulated for topical administration. The term “topical” as used herein includes any route of administration that enables the compounds to line the skin or mucosal tissues.

The formulation suitable for topical application may be in the form of, for example, cream, lotion, solution, gel, ointment, paste, plaster, paint, bioadhesive, or the like, and/or may be prepared so as to contain liposomes, micelles, microparticles and/or microspheres. The formulation may be aqueous, i.e., contain water, or may be non-aqueous and optionally used in combination with an occlusive overlayer so that moisture evaporating from the body surface is maintained within the formulation upon application to the body surface and thereafter.

Ointments, as is well known in the art of pharmaceutical formulation, are semisolid preparations that are typically based on petrolatum or other petroleum derivatives.

Formulations may also be prepared with liposomes, micelles, microparticles and/or microspheres. Liposomes are microscopic vesicles having a lipid wall comprising a lipid bilayer, and can be used as drug delivery systems. Micelles are known in the art to be comprised of surfactant molecules arranged so that their polar head groups form an outer spherical shell, while the hydrophobic, hydrocarbon chains are oriented towards the center of the sphere, forming a core. Microparticles are particulate carrier systems in the micron size range, normally prepared with polymers, which can be used as delivery systems for drugs or vaccines that are usually trapped within the particles. Microspheres, similarly, may be incorporated into the present formulations and drug delivery systems. Like liposomes and micelles, microspheres essentially encapsulate a drug or drug-containing formulation. Microspheres are generally, although not necessarily, formed from synthetic or naturally occurring biocompatible polymers, but may also be comprised of charged lipids such as phospholipids.

Preparations of formulations suitable for topical administration are well known in the art and described in the pertinent texts and literature.

In general, pharmaceutical compositions will comprise at least one of the peptides as defined herein together with a pharmaceutically acceptable carrier which will be well known to those skilled in the art. The compositions may further comprise for example, one or more suitable excipients, diluents, fillers, solubilizers, preservatives, stabilizers, carriers, salts, buffering agents and other materials well known in the art depending upon the dosage form utilized. Methods of composition are well known in the art.

In the present context, the term “pharmaceutically acceptable carrier” is intended to denote any material, which is inert in the sense that it substantially does not have any therapeutic and/or prophylactic effect per se and that are non-toxic. A pharmaceutically acceptable carrier may be added to the peptides as defined herein with the purpose of making it possible to obtain a pharmaceutical composition, which has acceptable technical properties.

Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of polypeptides include polysaccharides such as sodium carboxymethylcellulose or methylcellulose, polyvinylpyrrolidone, polyacrylates, polyoxyethylene-polyoxypropylene-block polymers, PEG, and wood wax alcohols.

Examples of stabilizer include an amino acid, such as for instance, glycine; or an oligosaccharide, such as for example, sucrose, tetralose, lactose or a dextran. Alternatively, the stabilizer may be a sugar alcohol, such as for instance, mannitol; or a combination thereof.

The salt or buffering agent may be any salt or buffering agent, such as for example, sodium chloride, or sodium/potassium phosphate, respectively. Preferably, the buffering agent maintains the pH of the pharmaceutical composition in the range of about 5.5 to about 7.5. The salt and/or buffering agent is also useful to maintain the osmolality at a level suitable for administration to a human or an animal.

The peptides used for in vivo administration should be sterile. This may be accomplished by filtration through sterile filtration membranes, prior to or following lyophilization and reconstitution. The peptides ordinarily will be stored in lyophilized form or in solution. Therapeutic peptide compositions generally are placed into a container having a sterile access port, for example, an intravenous solution bag or vial having a stopper pierceable by a hypodermic injection needle.

For use in the methods defined herein, the invention also provides an article of manufacture or a commercial package or kit, such as an FDA approved kit, which may comprise: a container, a label on the container and a composition comprising one or more unit dosage forms of the peptides of the present invention as active agent. The kit may be accompanied by instructions for dosage, administration and indications to be treated. The instructions may indicate that the composition is effective for, inter alia, protecting against oxidative stress-induced damage, reducing oxidative stress-induced damages, protecting against cell injuries induced by oxidative stress, protecting against ROS-induced cell injuries, inducing muscle regeneration, reducing functional impairment of muscle cells, inducing skeletal muscle regeneration, having antioxidant effect on oxidative-damaged cells and reducing functional impairment of skeletal muscle cells and protecting satellite cells from oxidative stress-induced damage.

An “effective amount” or a “therapeutically effective amount” refers to an amount effective, at dosages and for periods of time necessary, to achieve the desired therapeutic result. A therapeutically effective amount of the peptides noted herein may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as in protecting against oxidative stress-induced damage, reducing oxidative stress-induced damages, protecting against cell injuries induced by oxidative stress, protecting against ROS-induced cell injuries, inducing muscle regeneration, reducing functional impairment of muscle cells, inducing skeletal muscle regeneration, having antioxidant effect on oxidative-damaged cells and reducing functional impairment of skeletal muscle cells and protecting satellite cells from oxidative stress-induced damage. A prophylactically effective amount can be determined as described above for the therapeutically effective amount. For any particular subject, specific dosage regimens may be adjusted over time according to the individual need and the professional judgment of the person administering or supervising the administration of the compositions.

For example, a therapeutically effective amount or effective dose of the peptides as defined herein (also referred to herein as “active compound”) is an amount sufficient for in protecting against oxidative stress-induced damage, reducing oxidative stress-induced damages, protecting against cell injuries induced by oxidative stress, protecting against ROS-induced cell injuries, inducing muscle regeneration, reducing functional impairment of muscle cells, inducing skeletal muscle regeneration, having antioxidant effect on oxidative-damaged cells and reducing functional impairment of skeletal muscle cells and protecting satellite cells from oxidative stress-induced damage. The methods and/or assays for measuring such parameters are known to those of ordinary skill in the art.

The therapeutically effective amount of the invention will generally vary from about 0.001 μg/kg to about 100 mg/kg, more particularly from about 0.01 μg/kg to about 10 mg/kg, and even more particularly from about 1 μg/kg to about 1 mg/kg. Therapeutically effective amounts or effective doses that are outside this range but that have the desired therapeutic effect are also encompassed by the present invention.

In a further embodiment, the present polypeptides may be administered in combination with additional pharmacologically active substances or may be administered in combination with another therapeutic method. The combination may be in the form of a kit-in-part system, wherein the combined active substances may be used for simultaneous, sequential or separate administration.

### VI) Experiments and Data Analysis

### UAG Protects Against Ischemia-Mediated Functional Impairment in Skeletal Muscle

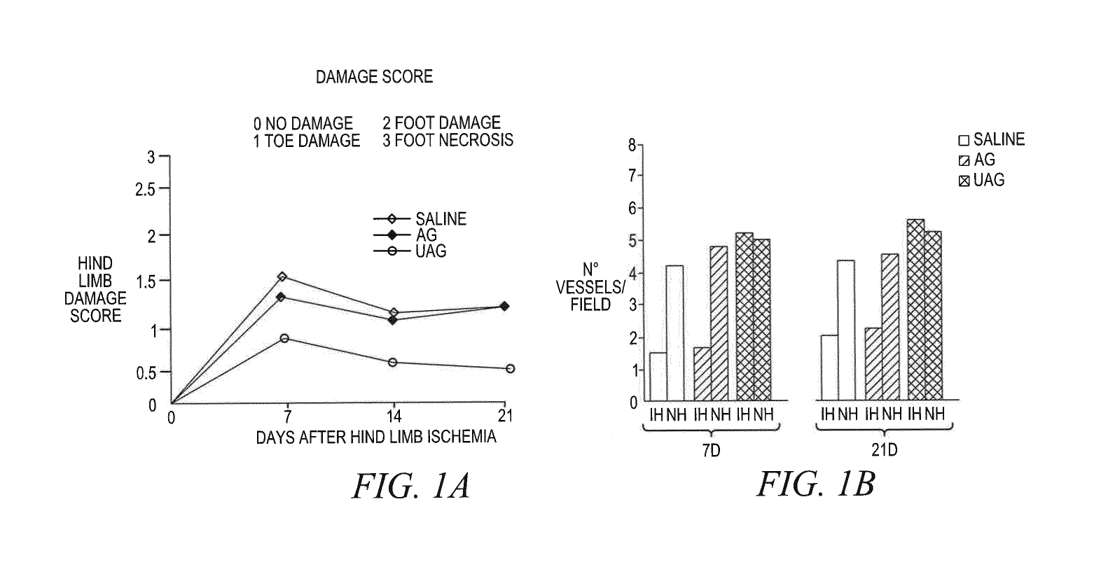

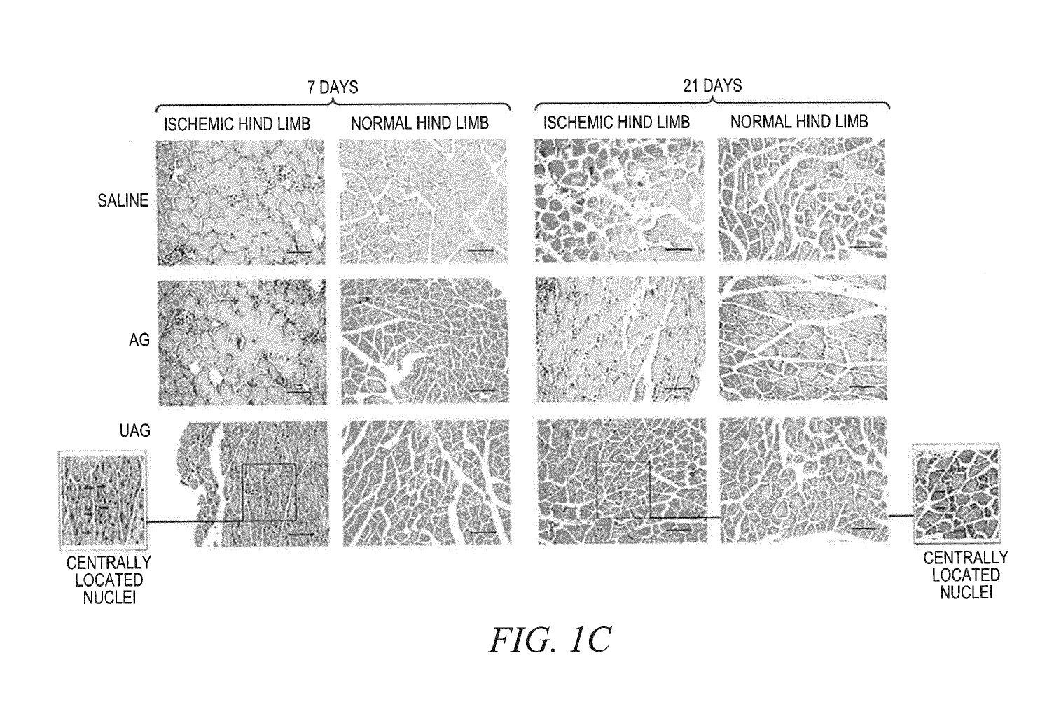

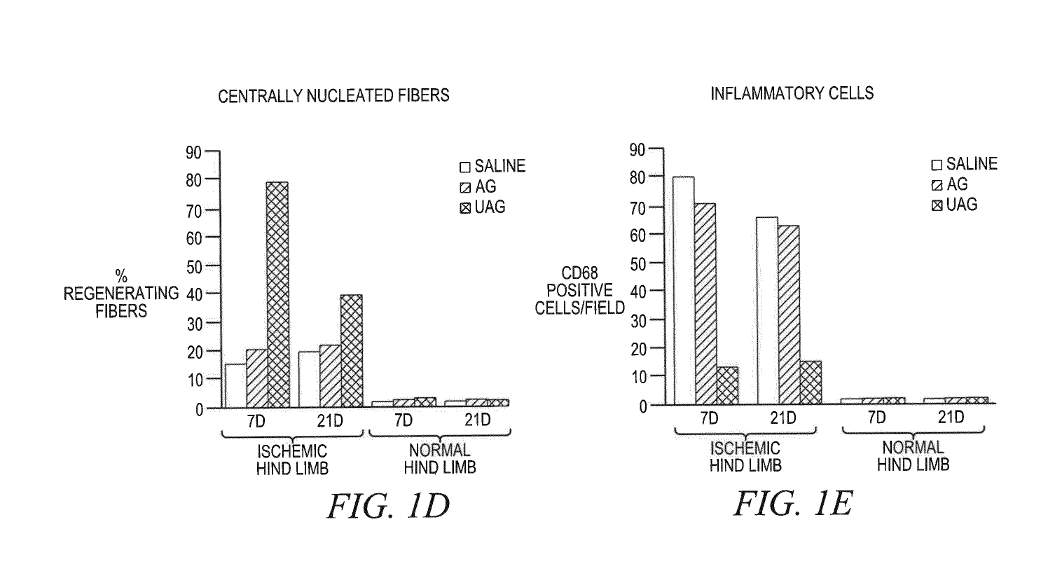

Unilateral hind-limb ischemia was induced in C57BL/6J mice, and mice were treated daily with either saline, AG, or UAG, beginning at day 0. When a functional score was applied, it was observed that the damage was significantly higher in saline- and AG-treated groups than in the UAG-treated group as shown in FIG. 1A. To assess whether these functional differences were due to differences in tissue reperfusion, the number of vessels was counted in ischemic muscles of treated animals. UAG-treated mice had a larger number of functional vessels compared to the other groups as shown in FIG. 1B. Furthermore, ischemic muscles from AG- and saline-treated mice had a significantly lower capillary density than in contralateral control muscles from the same animals, whereas no such differences were observed in UAG-treated animals (FIG. 1B). Analysis of tissue regeneration in gastrocnemius muscles revealed that muscles from UAG-treated mice contained an increased number of regenerating myofibers (FIGS. 1C and 1D). In addition, those mice had a reduced number of CD68-positive inflammatory cells (FIG. 1E). No changes were observed in the numbers of regenerating fibers or CD68-positive inflammatory cells in normo-perfused muscles (FIGS. 1C, 1D and 1E). Thus UAG but not AG appears to protect against ischemia-induced damage.

### UAG Increases the Number of Pax-7-Positive Cells

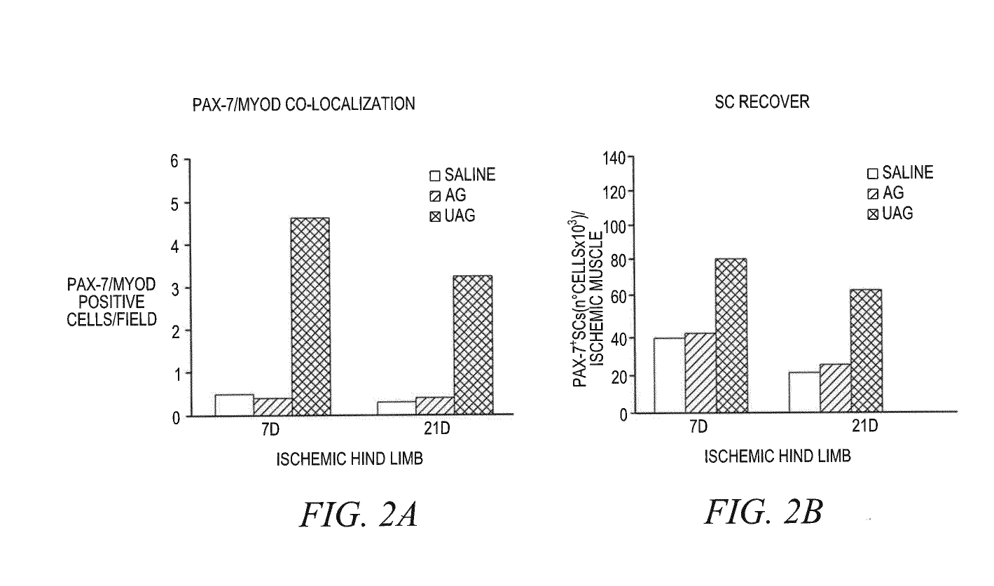

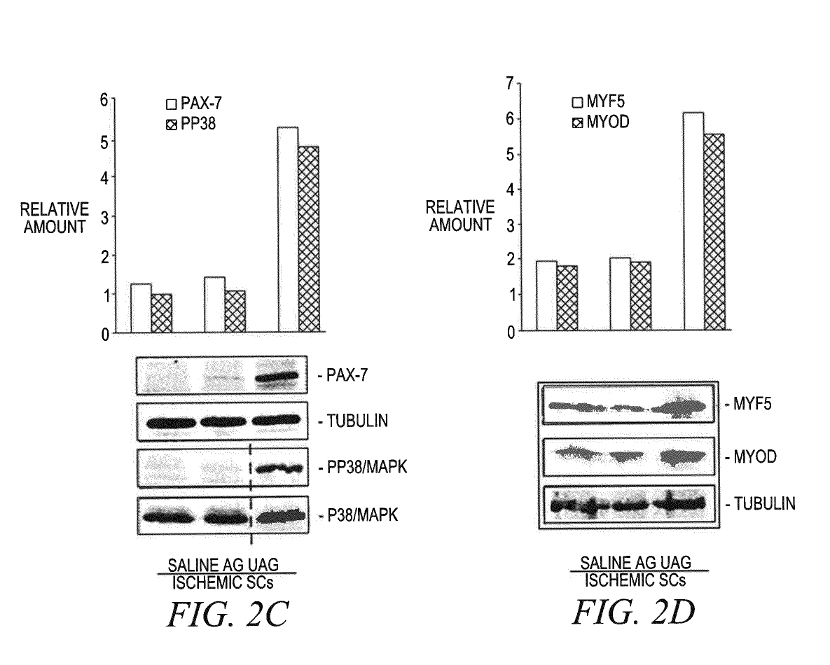

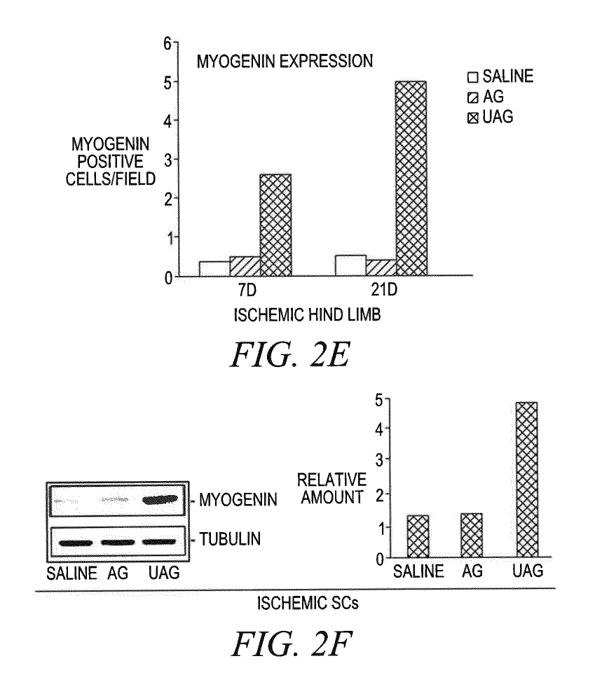

SMR in vivo depends on the expansion and differentiation of SCs³⁷ co-expressing Pax-7 and MyoD. Thus, the number of cells expressing both Pax-7 and MyoD was evaluated. At day 7, ischemic muscles from UAG-treated mice had an increased number of Pax-7/MyoD+ cells compared to ischemic muscles from AG-treated and control mice (FIG. 2A). At day 21, scattered Pax-7/MyoD+ cells were still present in ischemic muscles from UAG- but not AG- or saline-treated mice (FIG. 2A). SCs were also isolated and counted (FIG. 2B). This ex vivo evaluation revealed a significantly increased number of Pax-7/MyoD/Myf5+ SCs in UAG-treated mice compared to AG- and saline-treated animals (FIGS. 2B, 2C and 2D). Consistent with previous reports indicating that SC proliferation depends on p38/MAPK activation^{36,37}, levels of phospho(p)-p38/MAPK protein were found to be higher in SCs from UAG-treated mice than in those from saline- or AG-treated groups (FIG. 2C). Scattered Pax-7+/MyoD− cells were detected in normoperfused muscles in all groups. In addition, there were an increased number of myogenin-positive cells in ischemic muscles from UAG-treated mice (FIGS. 2E and 2F).

### UAG Induces SOD-2 Expression in SCs

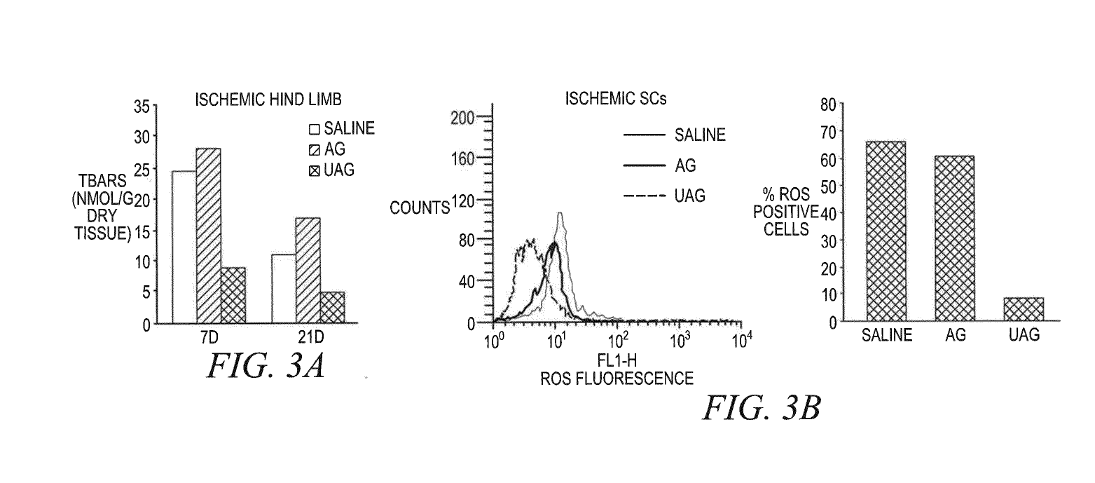

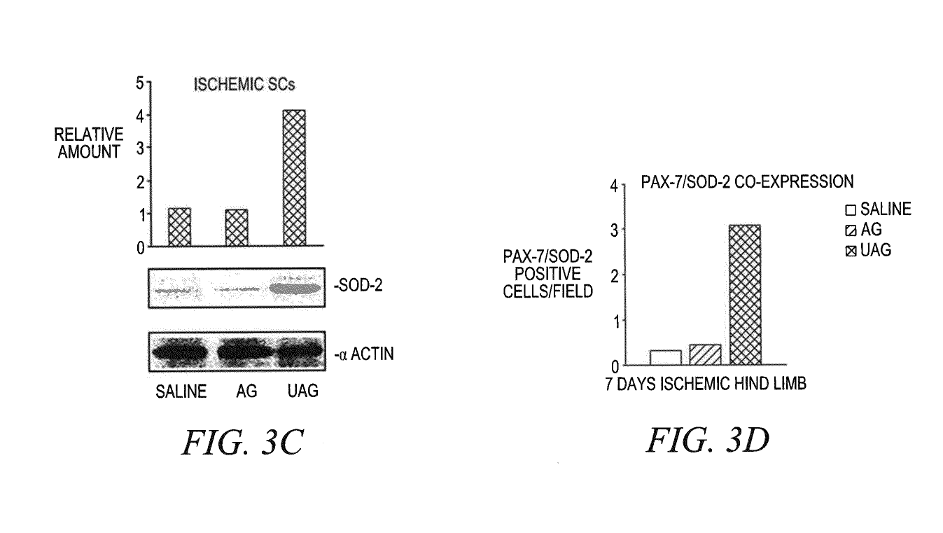

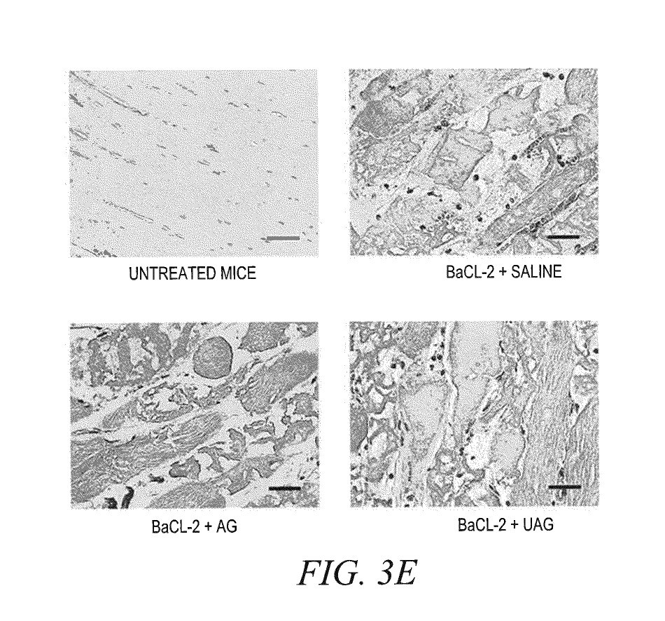

Thiobarbituric acid reactive substances (TBARS) were first evaluated in ischemic muscles. Significant increases in the concentration of TBARS were observed in AG- and saline- but not UAG-treated muscles (FIG. 3A). These data suggest that UAG may promote SC expansion by inducing an efficient antioxidant response. Since the mitochondria-specific antioxidant enzyme SOD-2 is known to be diminished in patients with PAD, the expression of SOD-2 expression and ROS content in SCs isolated from different groups were analyzed. It was found that ROS production was lower and SOD-2 protein expression higher in SCs from UAG-treated mice than from AG- or saline-treated animals (FIGS. 3B and 3C). Furthermore, double immunostaining (data not shown) of ischemic muscle from UAG-treated mice revealed an increased number of SOD-2/Pax-7+ cells compared to AG-treated mice and controls (FIG. 3D). Finally, when UAG was administered to mice treated with BaCl₂, which is known to induce ROS-independent damage³⁸, regeneration of skeletal muscle was no longer observed (FIG. 3E). This experiment and these data clearly demonstrate that UAG specifically acts on ROS-mediated damage, but not on toxic-mediated damage. UAG thus appears to influence SMR via an antioxidant effect.

Together, these data suggest that UAG may represent a defense mechanism against ROS and that diseases characterized by mitochondrial dysfunction and increased ROS generation, may likely also benefit from UAG treatment.

### UAG-Induced SC Cell-Cycle Entry is Recapitulated In Vitro Even in Mice Lacking the Entire Ghrelin System

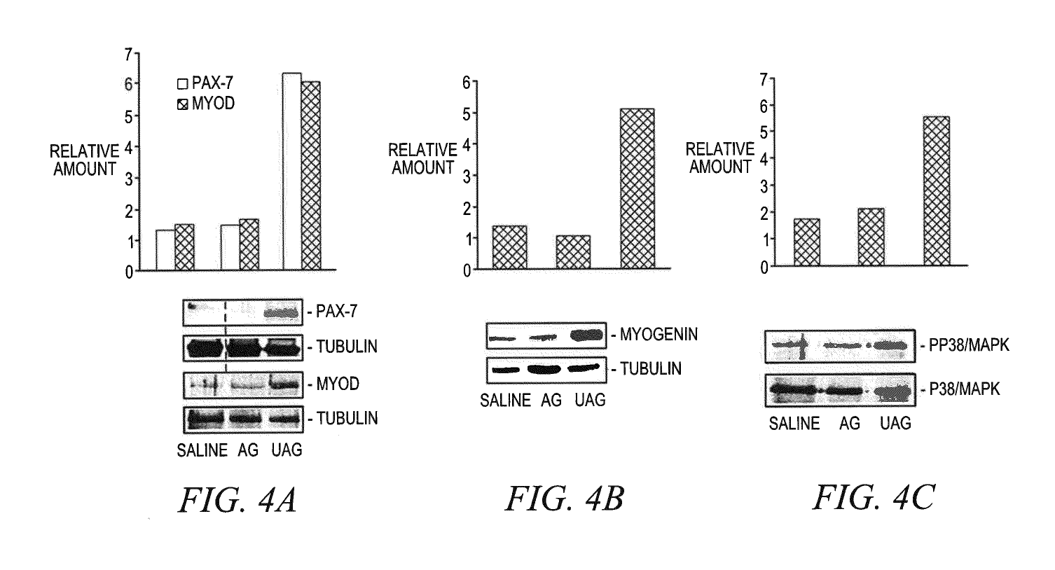

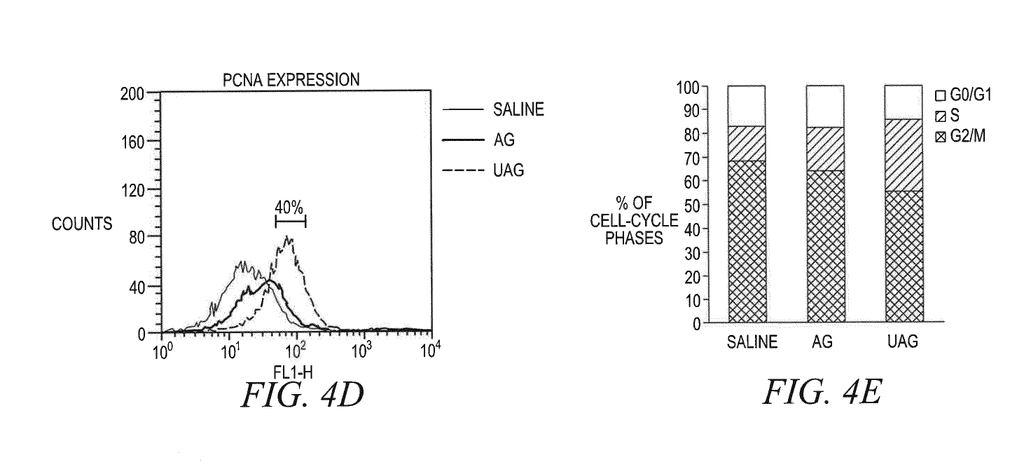

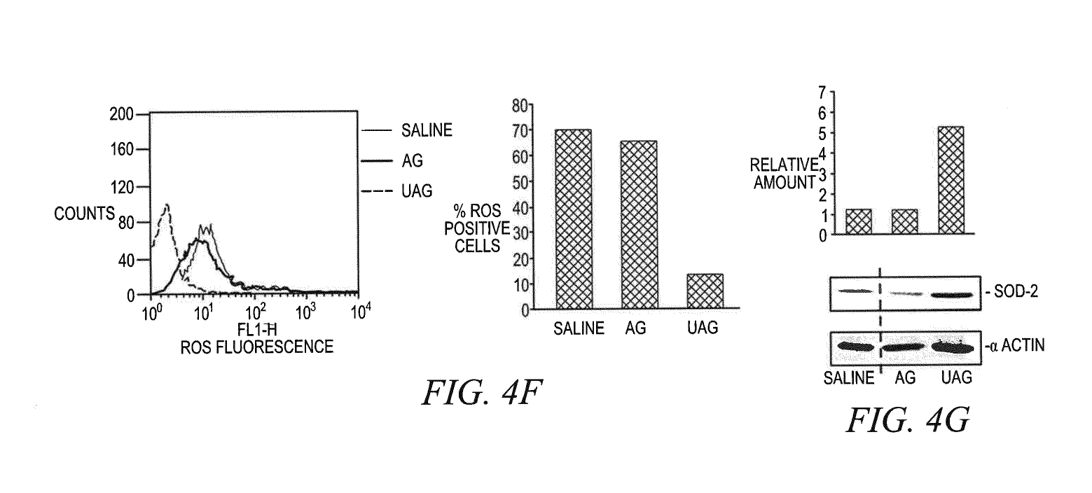

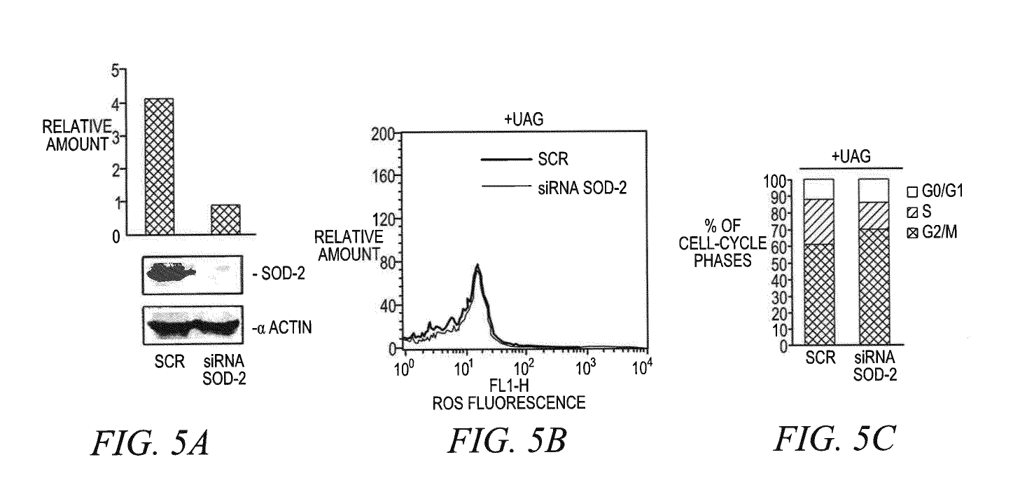

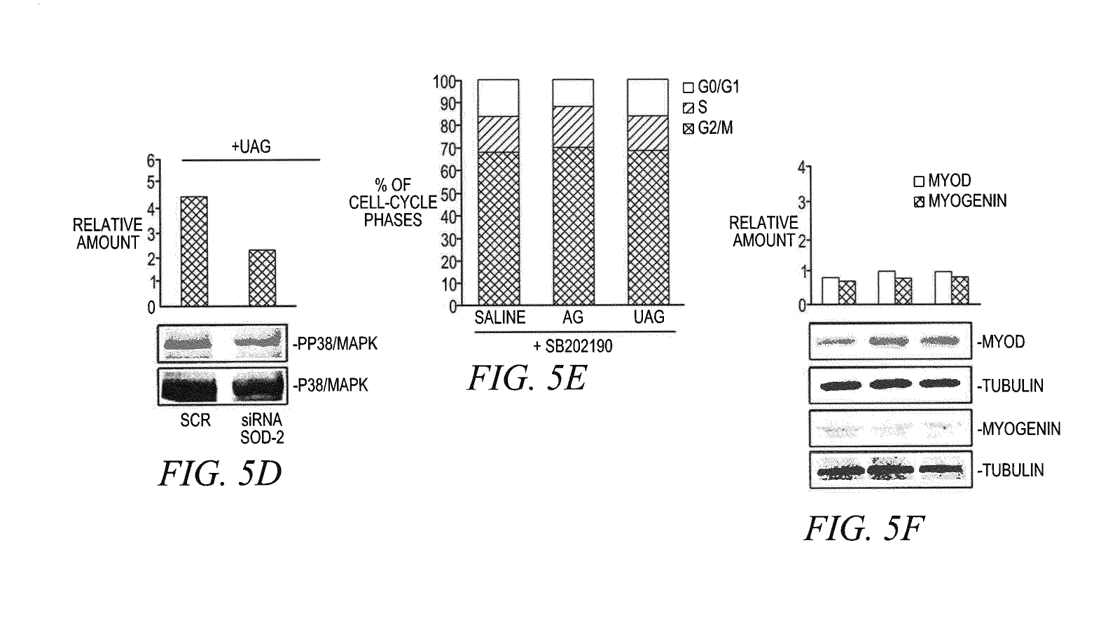

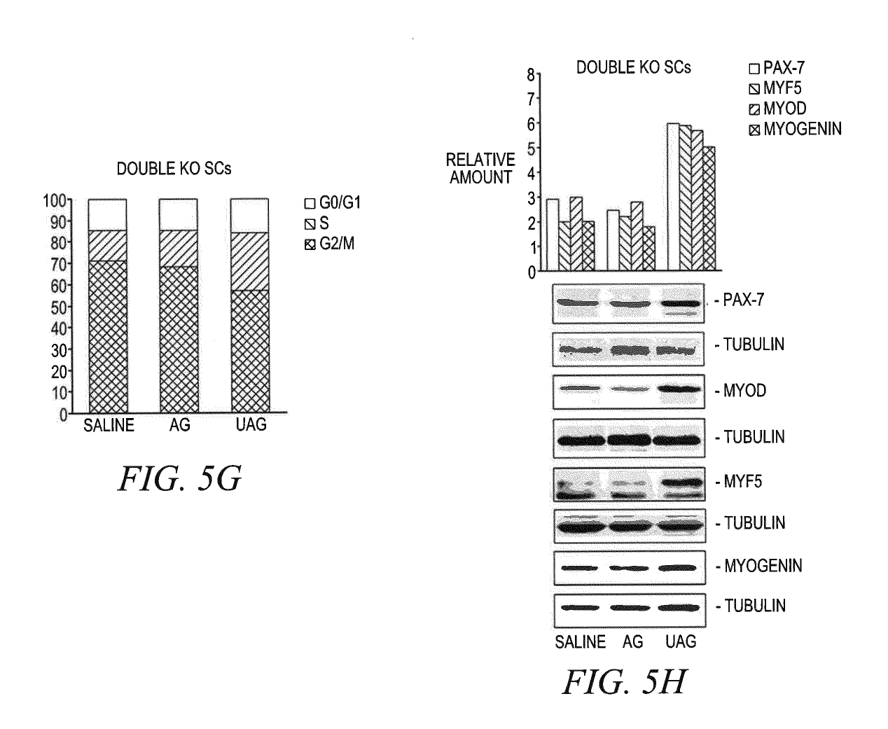

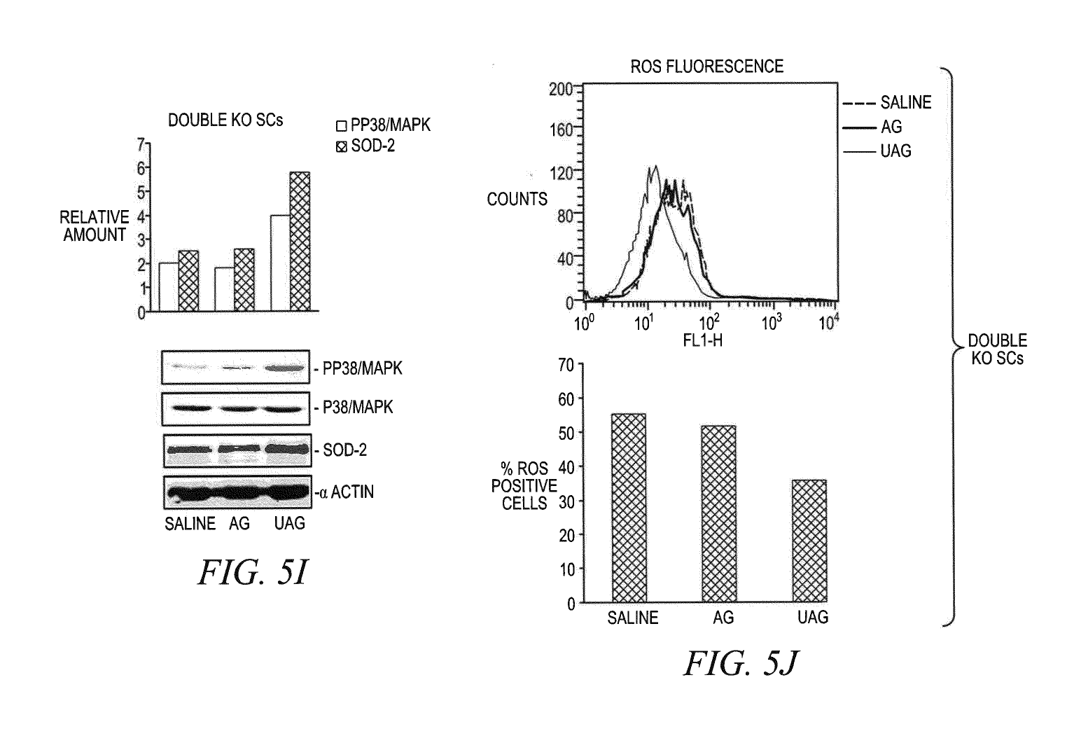

Primary SCs recovered from normo-perfused muscles were subjected to in vitro ischemia and evaluated for cell-cycle progression upon treatment. Again, only UAG challenge induced expression of Pax-7, MyoD (FIG. 4A) and myogenin (FIG. 4B), and increased levels of pp 38/MAPK (FIG. 4C). In addition, UAG challenge increased PCNA expression (FIG. 4D) and the number of cells in S phase (FIG. 4E). When examined under the same experimental conditions, ROS production was decreased (FIG. 4F) and SOD-2 expression increased (FIG. 4G) following UAG treatment. When SOD-2 was silenced in SCs using siRNA (FIG. 5A) and subjected to in vitro ischemia, UAG did not protect SCs against ROS generation (FIG. 5B), did not induce p38/MAPK phosphorylation (FIG. 5D) and the cells did not undergo cell-cycle progression (FIG. 5C). Moreover, addition of SB202190, an inhibitor of p38/MAPK phosphorylation, blocked cell-cycle entry and prevented MyoD and myogenin expression in SCs exposed to UAG (FIGS. 5E and 5F). These data indicate that, after ischemia, SOD-2 expression is important for UAG-induced p38/MAPK phosphorylation leading to cell-cycle entry in SCs.

To assess whether the effects of UAG on SMR and SC cell-cycle entry occurred through the classic ghrelin signaling pathway, mice lacking the GHSR1a and ghrelin genes³⁹ was analyzed. SCs from these double KO mice were subjected to in vitro ischemia in the presence of AG or UAG. Once again, only UAG promoted SC cell-cycle progression (FIG. 5G) and induced p-p38/MAPK, Pax-7, MyoD, Myf5 and myogenin expression (FIGS. 5H and 5I). Moreover, unlike AG, UAG protected SCs from ROS generation and induced SOD-2 expression (FIGS. 5I and 5J). These findings, along with the failure to detect in vivo effects of AG, further support the possibility that UAG induces AG-independent activities via specific binding sites.

miR-221 and miR-222 Control UAG-Induced SC Cell-Cycle Entry by Regulating the Expression of p57^{kip}

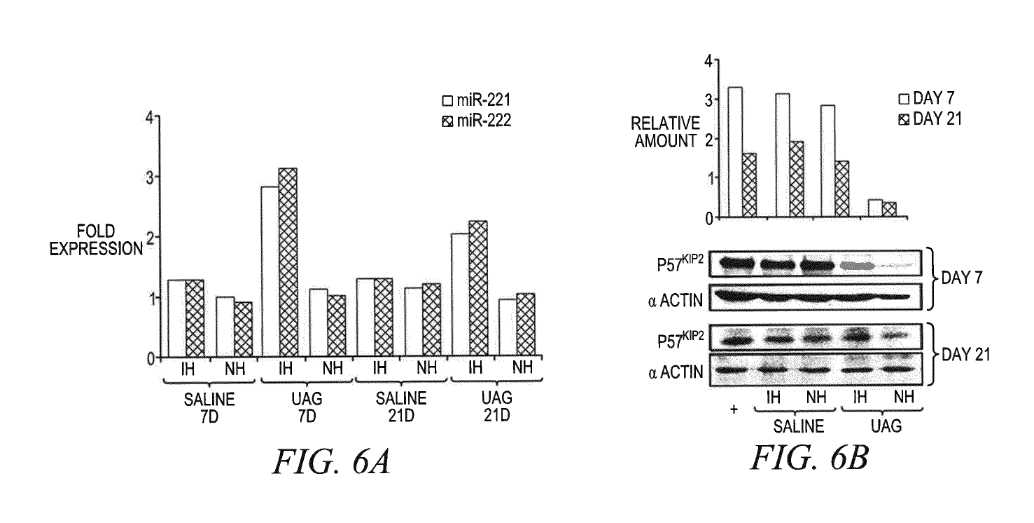

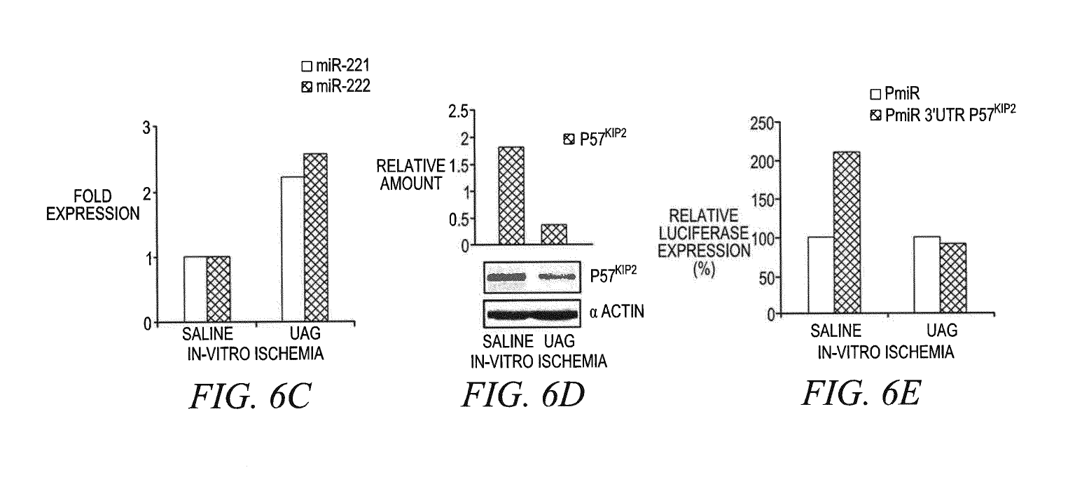

To address the mechanism through which UAG exerts its effects, the expression of miR-221 and miR-222, recently emerged as important regulators of myogenesis^{40,41}, was analyzed. Expression of miR-221 and miR-222 was significantly increased in SCs recovered from muscles of UAG-treated mice compared to controls (FIG. 6A). The expression of p57^{kip2}, a known target gene of miR-221/22241, was therefore analyzed and it was found that levels of p57^{kip2} protein were reduced in SCs from UAG-treated mice (FIG. 6B). Similar results were obtained when SCs recovered from normoperfused muscles were subjected to in vitro ischemia and UAG (FIGS. 6C and 6D). These data were confirmed by luciferase assay (FIG. 6E). Furthermore, in loss-of-function experiments involving transfection of SCs with anti-miR-221/222 antago-miRs, UAG no longer had any effect on cell-cycle entry or expression of SOD-2, Pax-7, MyoD, or myogenin in SCs (data not shown). The observation that p-p38/MAPK levels were reduced under these experimental conditions (data not shown) provides further evidence that both miRs are important mediators of SC cell-cycle entry.

miR-221/222 Expression is Modulated by Oxidative Stress and is Important for SMR Upon Ischemia

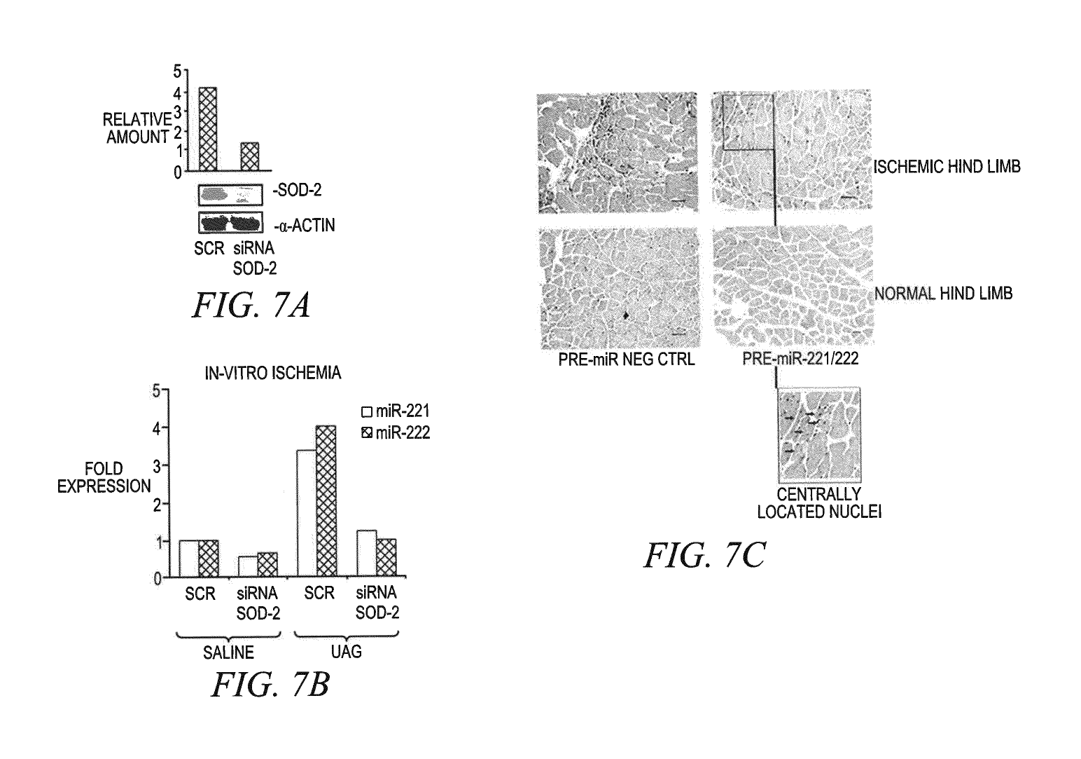

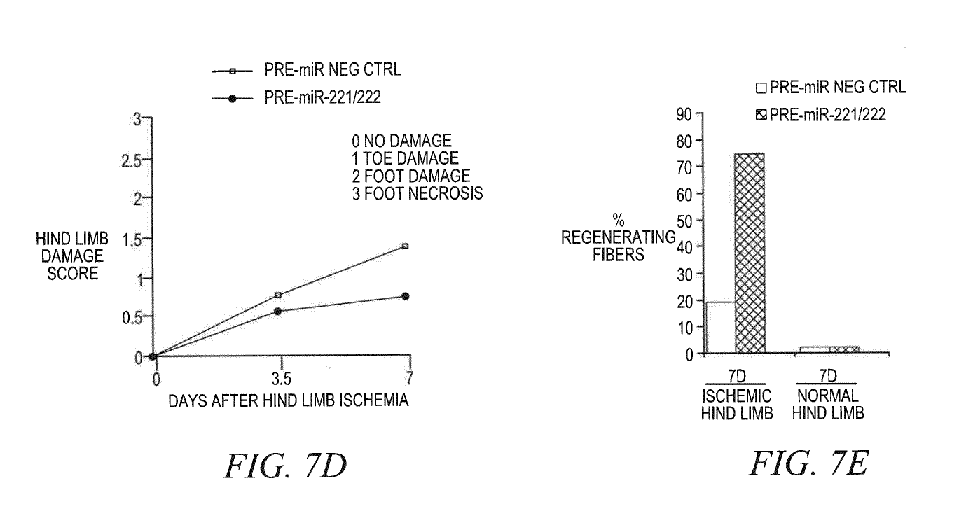

Analysis of miR-221/222 expression in the in vitro model of ischemia following SOD-2 depletion (FIG. 7A) revealed that SOD-2 knock-down prevents UAG-induced miR-221/222 expression (FIG. 7B). Thus, miR-221/222 expression appears to be modulated by ROS generation. The in vivo role of miR-221/222 in SMR was analyzed by injection of pre-miR-221/222 in the herein discussed model. Under these conditions, pre-miR-221/222 injection led to lower damage scores and significant myofiber regeneration (FIGS. 7C, 7D and 7E) even in the absence of UAG. Furthermore, SCs recovered from those mice had high levels of miR-221/222 expression and increased levels of MyoD, myogenin and p-p38/MAPK protein (data not shown).

UAG, Fragments and Analogs Thereof Protect C2C12 Mouse Muscular Cell Line from Oxidative Stress

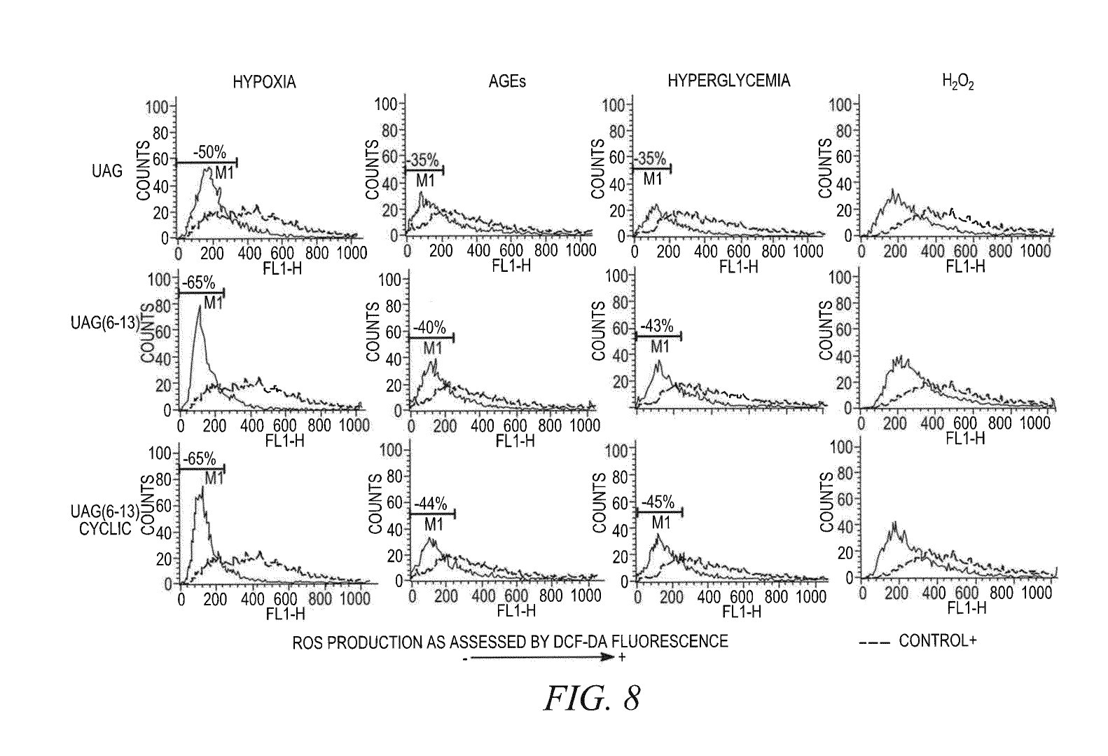

The effect of UAG and UAG fragments on C2C12 mouse muscular cell line subjected to oxidative stress was assessed. In this analysis, oxidative stress was caused by hypoxia, AGEs, hyperglycemia or by H₂O₂. The results obtained demonstrate that UAG, UAG (6-13) and UAG(6-13)cyclic have a protective effect on C2C12 cells against oxidative stress (FIG. 8).

### VII) Materials and Technical Protocols

Murine Hind-Limb Ischemia Model

Male C57BL/6J mice (Charles River Lab., Wilmington, Mass., USA) were anesthetized and unilateral hind limb ischemia was induced as described⁴². The normo-perfused contralateral limb of each mouse was used as an internal control. After hind-limb ischemia, animals (18 mice per group) were treated by intra-peritoneal injection daily from 0 to day 21 with either saline, AG (100 4/kg) or UAG (100 4/kg). In selected experiments, mice received intramuscular injections of pre-miR oligonucleotides (5 mice/group). To induce toxic damage, 100 μl of 1% barium chloride (BaCl₂, Sigma Aldrich) was injected unilaterally into the hind limb (9 mice). Mice were treated according to European Guidelines and policies as approved by the University of Turin Ethical Committee.

In-Vivo Assessment of Limb Function

Semiquantitative estimation (by repeated measures analyzed with ANOVA and Newman-Keuls Multiple Comparison test) of foot damage was performed serially using the following classification: 3=dragging of foot (foot necrosis), 2=no dragging but no plantar flexion (foot damage), 1=plantar flexion but no toe flexion (toe damage), and 0=flexing the toes to resist gentle traction on the tail (no damage)⁴³.

### Histological and Immunofluorescence (IF) Analysis

Gastrocnemius muscle sections from ischemic or normo-perfused limbs were stained with hematoxylin and eosin for histological analysis. The proportion of fibers with central nuclei (regenerating fibers) was measured by MetaMorph software (Life Sciences Research Imaging Systems) in the injured area and the cross-sectional areas of the fibers in the injured and non-injured areas. For IF analysis, muscle sections were processed as described previously⁴⁴. The number of cells expressing the indicated markers or CD31 positive vessels was evaluated as previously described³⁴.

Cell Cultures and In-Vitro Ischemia

SCs were isolated from gastrocnemius muscles of C57BL/6J wild type mice subjected to ischemia or C57BL/6J mice lacking the GHSR1a and ghrelin genes (10 mice, kind gift of Professor M. Tschöp)³⁹. To obtain SCs, muscle samples were subjected to enzymatic digestion as described⁴⁵. In selected experiments, SCs were recovered from normo-perfused muscles and subjected to in-vitro ischemia in presence of saline, AG (1 μmol/L) or UAG (1 μmol/L). In-vitro ischemia was induced by incubating cells in DMEM+2% FCS at 5% CO₂/95% N₂ humidified atmosphere, yielding 1% O₂ concentrations for 24 h¹⁸. The in-vitro ischemia was also performed in the presence of SB202190 (1 μmol/L).

Cell-Cycle Progression and Proliferation

SC cell-cycle progression was evaluated by evaluating the percentage of PCNA-positive cells or by FACS analysis as previously described⁴⁶. The percentage of cells in each cell cycle phase was determined by ModFit LT software (Verity Software House. Inc, topsham, ME, USA). Celss proliferation was also assayed by evaluating the percentage of PCNA-positive cells by FACS analysis.

Western Blot (WB) Analysis

Cells were lysed and protein detection was obtained as previously described⁴⁷. Cells were lysed (50 mmol/L Tris HCl [pH 8.3], 1% Triton X-100, 10 mmol/L PMSF, 100 U/ml aprotinin, 10 μmol/L leupeptin) and protein concentrations were obtained as previously described⁴⁷. Proteins (50 μg) were subjected to SDS-PAGE, transferred into nitrocellulose membrane, blotted with the indicated antibodies and revealed by chemiluminescence detection system (ECL). Densitometric analysis was used to calculate the differences in the fold induction of protein levels and normalized to tubulin, a actin or p38MAPK content. Values are reported as relative amount

Oxidative Stress Measurement

Intracellular ROS production was evaluated using DCF-DA (5-(and-6)-carboxy-2′,7′-dichlorofluorescein diacetate, 0.5 Lmol/L final concentration) (Molecular Probe, Invitrogen) assay as previously described²³. The formation of TBARS was determined in muscles using the OXI-TEK kit (ZeptoMetrix Corp.) and a luminescence spectrometer (Bio-Rad Laboratories, Hercules, Calif.) with excitation set at 530 nm, emission at 550 nm to measure in-vivo oxidative stress levels³³.

RNA Isolation and Quantitative Real-Time PCR (qRT-PCR) for miRNAs

Total RNA was isolated using TRIzol reagent (Invitrogen) from SCs recovered from muscles of treated animals or from SCs subjected to in-vitro ischemia. miR-221/222 expression was evaluated by qRT-PCR as previously described⁴⁸. Loss-of-function experiments were performed in SCs transfected for 48 h with anti-miRNA negative control, anti-miR-221/222 antagonists (Applied Biosystem, Foxter Cyto CA, USA), according to the manufacturer's instructions⁴⁸.

SOD-2 Silencing by Small Interfering RNAs (siRNA)

To obtain SOD-2 inactivation, SCs were transiently transfected with siRNA for SOD-2 or with duplex siRNAs (Qiagen, Valencia, Calif., USA) as previously described⁴⁴. Transfection was performed according to the manufacturer's instructions. Whole cell extracts were processed 48 h after transfection. Cell viability was evaluated at the end of each experiment.

Luciferase miRNA Target Reporter Assay

The luciferase reporter assay was performed using a construct generated by subcloning the PCR products amplified from the full-length 3′UTR of p57Kip2 as previously described⁴⁷.

In-Vivo Gain of Function Analysis

To evaluate the effects of miR-221/222 expression in-vivo, a combination of pre-miR-221/222 or pre-miR negative control (50 μl of 50 nM stock solution of pre-miR oligonucleotides into 12 μl of Optifect, Invitrogen) was injected directly into the ischemic gastrocnemius muscle of C57BL/6J mice. Pre-miRs or controls were administrated 3 times a week. At day 7, animals were sacrificed and tissues were recovered and processed as described above for histological analysis. SCs were also isolated and evaluated by WB for the indicated markers and by qRT-PCR for miRNA expression.

Statistical Analysis

Between-group comparisons were carried out by t test. Comparisons between 3 or more groups were performed by one-way ANOVA and significance was evaluated using the Newman-Keuls multi-comparison post hoc test. The cutoff for statistical significance was set at P<0.05. All statistical analyses were carried out with Graph Pad Prism version 5.04 (Graph Pad Software, Inc, USA).

Oxidative Stress Measurement

Kinetic analysis of ROS production was evaluated by using DCF-DA (5-(and-6)-carboxy-2′,7′-dichlorofluorescein diacetate, 0.5 μmol/L final concentration) (Molecular Probe, Invitrogen) assay. C2C12 cells were cultured with 400 μg/ml Advanced Glycated End-product (AGE) or 25 mM glucose (high glucose HG) for 48 h, H₂O₂ (100 μM) for 2 h. In parallel experiments C2C12 cells were subjected to hypoxia (DMEM+2% FCS in a 5% CO₂-95% N₂ humidified atmosphere, yielding to 1% O₂ concentrations for 24 h). UAG (1 μmol/L) AZP 531 (1 μmol/L) or AZP-502 (1 μmol/L) was added where indicated.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

All documents mentioned in the specification are herein incorporated by reference.

### VIII) REFERENCE LIST

1. Kojima M, Hosoda H, Date Y, Nakazato M, Matsuo H and. Kangawa K; Ghrelin is a growth-hormone-releasing acylated peptide from stomach, Nature 402:656-660 (1999).
2. Gnanapavan S, Kola B, Bustin S A, Morris D G, McGee P, Fairclough P, Bhattacharya S, Carpenter R, Grossman A B and Korbonits M; The tissue distribution of the mRNA of ghrelin and subtypes of its receptor, GHS-R, in humans, J. Clin. Endocrinol. Metab. 87:2988-2991 (2002).
3. Howard A D, Feighner S D, Cully D F, Arena J P, Liberator P L, Rosenblum C I, Hamelin M, Hreniuk D L, Palyha O C, Anderson J, Paress P S, Diaz C, Chou M, Liu K K, McKee K-K, Pong S-S, Chaung L-Y, Elbrecht A, Dashkevicz M, Heavens R, Rigby M, Sirinathsinghji D, Dean D C, Melillo D G, Patchett A A, Nargund R, Griffin P R, DeMartino J A, Gupta S K, Schaeffer J A, Smith R G, Van der Ploeg L H T; A receptor in pituitary and hypothalamus that functions in growth hormone release. Science 273:974-977 (1996).
4. Ariyasu H, Takaya K, Tagami T, Ogawa Y, Hosoda K, Akamizu T, Suda M, Koh T, Natsui K, Toyooka S, Shirakami G, Usui T, Shimatsu A, Doi K, Hosoda H, Kojima M, Kangawa K, Nakao K.; Stomach is a major source of circulating ghrelin, and feeding state determines plasma ghrelin-like immunoreactivity levels in humans. J Clin Endocrinol Metab. October; 86(10):4753-8 (2001)
5. Date Y, Murakami N, Toshinai K, Matsukura S, Niijima A, Matsuo H, Kangawa K, Nakazato M. The role of the gastric afferent vagal nerve in ghrelin-induced feeding and growth hormone secretion in rats. Gastroenterology 123(4):1120-1128, 2002.
6. Kojima M, Hosoda H, Date Y, Nakazato M, Matsuo H, Kangawa K. Ghrelin is a growth-hormone-releasing acylated peptide from stomach. Nature 402(6762):656-660, 1999.
7. Tschop M, Smiley D L, Heiman M L. Ghrelin induces adiposity in rodents. Nature 407(6806):908-913, 2000.
8. Broglio F, Gianotti L, Destefanis S, Fassino S, Abbate Daga G, Mondelli V, Lanfranco F, Gottero C, Gauna C, Hofland L, Van Der Lely A J, Ghigo E. The endocrine response to acute ghrelin administration is blunted in patients with anorexia nervosa, a ghrelin hypersecretory state. Clin Endocrinol (Oxf) 60(5):592-599, 2004.
9. Tong J, Prigeon R L, Davis H W, Bidlingmaier M, Kahn S E, Cummings D E, Tschöp M H, D'Alessio D. Ghrelin suppresses glucose-stimulated insulin secretion and deteriorates glucose tolerance in healthy humans. Diabetes September; 59(9):2145-51 (2010).
10. van der Lely A J, Tschop M, Heiman M L, Ghigo E; Biological, physiological, pathophysiological, and pharmacological aspects of ghrelin. Endocr Rev 25:426-457 (2004).
11. Asakawa A, Inui A, Fujimiya M, Sakamaki R, Shinfuku N, Ueta Y, Meguid M Mi, Kasuga M. Stomach regulates energy balance via acylated ghrelin and desacyl ghrelin. Gut. January; 54(1):18-24 (2005).
12. Broglio F, Gottero C, Prodam F, Gauna C, Muccioli G, Papotti M, Abribat T, Van Der Lely A J, Ghigo E. Non-acylated ghrelin counteracts the metabolic but not the neuroendocrine response to acylated ghrelin in humans. J Clin Endocrinol Metab. June; 89(6):3062-5 (2004).
13. Gauna C, Meyler F M, Janssen J A, Delhanty P J, Abribat T, van Koetsveld P, Hofland L J, Broglio F, Ghigo E, van der Lely A J. Administration of acylated ghrelin reduces insulin sensitivity, whereas the combination of acylated plus unacylated ghrelin strongly improves insulin sensitivity. J Clin Endocrinol Metab. October; 89(10):5035-42 (2004).
14. Kiewiet R M, van Aken M O, van der Weerd K, Uitterlinden P, Themmen A P, Hofland L J, de Rijke Y B, Delhanty P J, Ghigo E, Abribat T, van der Lely A J. Effects of acute administration of acylated and unacylated ghrelin on glucose and insulin concentrations in morbidly obese subjects without overt diabetes. Eur J Endocrinol 161:567-573 (2009).
15. Gauna C, Delhanty P J D, Hofland L J, Janssen J, Broglio F, Ross R J M, Ghigo E, van der Lely A J. Ghrelin stimulates, whereas des-octanoyl ghrelin inhibits, glucose output by primary hepatocytes. Journal of Clinical Endocrinology and Metabolism 90:1055-1060 (2005).
16. Gauna C, Kiewiet R M, Janssen J A, van de Zande B, Delhanty P J, Ghigo E, Hofland L J, Themmen A P, van der Lely A J. Unacylated ghrelin acts as a potent insulin secretagogue in glucose-stimulated conditions. Am J Physiol Endocrinol Metab 293:E697-704 (2007).
17. Zhao W, Diz D I, Robbins M E; Oxidative damage pathways in relation to normal tissue injury. Br J. Radiol. 80:S23-31 (2007).
18. Filigheddu N, Gnocchi V F, Coscia M, et al. Ghrelin and des-acyl ghrelin promote differentiation and fusion of C2C12 skeletal muscle cells. Mol Biol Cell. 2007; 18:986-994.
19. Baldanzi G, Filigheddu N, Cutrupi S, et al. Ghrelin and des-acyl ghrelin inhibit cell death in cardiomyocytes and endothelial cells through ERK1/2 and PI 3-kinase/AKT. J Cell Biol 0.2002; 159:1029-1037.
20. Gauna C, Delhanty P J, Hofland L J, Janssen J A, Broglio F, Ross R J, Ghigo E, van der Lely A J. Ghrelin stimulates, whereas des-octanoyl ghrelin inhibits, glucose output by primary hepatocytes. J Clin Endocrinol Metab. 2005; 90:1055-1060.
21. Soares J B, Leite-Moreira A F. Ghrelin, des-acyl ghrelin and obestatin: three pieces of the same puzzle. Peptides. 2008; 29:255-1270.
22. Lear P V, Iglesias M J, Feijoo-Bandin S, Rodriguez-Penas D, Mosquera-Leal A, Garcia-Rua V, Gualillo O, Ghe C, Arnoletti E, Muccioli G., Dieguez C, Gonzalez-Juanatey J R, Lago F. Des-acyl ghrelin has specific binding sites and different metabolic effects from ghrelin in cardiomyocytes. Endocrinology. 2010; 151:3286-3298.
23. Togliatto G, Trombetta A, Dentelli P, Baragli A, Rosso A, Granata R, Ghigo D, Pegoraro L, Ghigo E, Brizzi M F. Unacylated ghrelin rescues endothelial progenitor cell function in individuals with type 2 diabetes. Diabetes. 2010; 59:1016-1025.
24. Granata R, Settanni F, Julien M, Nano R, Togliatto G, Trombetta A, Gallo D, Piemonti L, Brizzi M F, Abribat T, van Der Lely A J, Ghigo E. Des-acyl ghrelin fragments and analogues promote survival of pancreatic β-cells and human pancreatic islets and prevent diabetes in streptozotocin-treated rats. J Med. Chem. 2012; 55:2585-2596.
25. Weitz J I, Byrne J, Clagett G P, Farkouh M E, Porter J M, Sackett D L, Strandness D E Jr, Taylor L M. Diagnosis and treatment of chronic arterial insufficiency of the lower extremities: a critical review. Circulation. 1996; 94:3026-3049.
26. Gray B H, Conte M S, Dake M D, Jaff M R, Kandarpa K, Ramee S R, Rundback J, Waksman R, American Heart Association Writing Group 7. Atherosclerotic Peripheral Vascular Disease Symposium II: lower-extremity revascularization: state of the art. Circulation. 2008; 118:2864-2872.
27. Cieri E, Lenti M, De Rango P, Isernia G, Marucchini A, Cao P. Functional ability in patients with critical limb ischaemia is unaffected by successful revascularisation. Eur J Vasc Endovasc Surg. 2011; 41:256-263.
28. Pipinos I I, Judge A R, Selsby J T, Zhu Z, Swanson S A, Nella A A, Dodd S L. The myopathy of peripheral arterial occlusive disease: Part 2. Oxidative stress, neuropathy, and shift in muscle fiber type. Vasc Endovascular Surg. 2008; 42:101-112.
29. Bhat H K, Hiatt W R, Hoppel C L, Brass E P. Skeletal muscle mitochondrial DNA injury in patients with unilateral peripheral arterial disease. Circulation. 1999; 99:807-812.
30. Finkel T. Radical medicine: treating ageing to cure disease. Nat Rev Mol Cell Biol. 2005; 6:971-976.
31. Madamanchi N R, Runge M S. Mitochondrial Dysfunction in Atherosclerosis. Circ. Res. 2007; 100:460-473.
32. Zaccagnini G, Martelli F, Fasanaro P, Magenta A, Gaetano C, Di Carlo A, Biglioli P, Giorgio M, Martin-Padura I, Pelicci P G, Capogrossi M C. p66ShcA modulates tissue response to hindlimb ischemia. Circulation. 2004; 109:2917-2923.
33. Zaccagnini G, Martelli F, Magenta A, Cencioni C, Fasanaro P, Nicoletti C, Biglioli P, Pelicci P G, Capogrossi M C. p66(ShcA) and oxidative stress modulate myogenic differentiation and SMR after hind limb ischemia. J Biol. Chem. 2007; 282:31453-31459.
34. Pedersen B L, Baekgaard N, Quistorff B. Muscle mitochondrial function in patients with type 2 diabetes mellitus and peripheral arterial disease: implications in vascular surgery. Eur J Vasc Endovasc Surg. 2009; 38:356-364.
35. Loffredo L, Marcoccia A, Pignatelli P, Andreozzi P, Borgia M C, Cangemi R, Chiarotti F, Violi F. Oxidative-stress-mediated arterial dysfunction in patients with peripheral arterial disease Eur Heart J. 2007; 28:608-612.
36. Jones N C, Tyner K J, Nibarger L, Stanley H M, Cornelison D D, Fedorov Y V, Olwin B B. The p38alpha/beta MAPK functions as a molecular switch to activate the quiescent satellite cell. J Cell Biol. 2005; 169:105-116.
37. Troy A, Cadwallader A B, Fedorov Y, Tyner K, Tanaka K K, Olwin B B. Coordination of satellite cell activation and self-renewal by Par-complex-dependent asymmetric activation of p38α/β MAPK. Cell Stem Cell. 2012; 11: 541-553.
38. Lu H, Huang D, Ransohoff R M, Zhou L. Acute skeletal muscle injury: CCL2 expression by both monocytes and injured muscle is required for repair. FASEB J. 2011; 25:3344-3355.
39. Kirchner H, Tong J, Tschop M H, Pfluger P T. Ghrelin and PYY in the regulation of energy balance and metabolism: lessons from mouse mutants. Am J Physiol Endocrinol Metab. 2010; 298:E909-919.
40. Cardinali B, Castellani L, Fasanaro P, Basso A, Alema S, Martelli F, Falcone G. Microrna-221 and microrna-222 modulate differentiation and maturation of skeletal muscle cells. PLoS One. 2009; 4:e7607.

41. Greco S, Perfetti A, Fasanaro P, Cardani R, Capogrossi M C, Meola G, Martelli F. Deregulated microRNAs in myotonic dystrophy type 2. PLoS One. 2012; 7:e39732.
42. Hellingman A A, Bastiaansen A J, de Vries M R, Seghers L, Lijkwan M A, Lowik C W, Hamming J F, Quax P H. Variations in surgical procedures for hind limb ischaemia mouse models result in differences in collateral formation. Eur J Vasc Endovasc Surg. 2010; 40:796-803.
43. Bosch-Marce M, Okuyama H, Wesley J B, et al. Effects of aging and hypoxia-inducible factor-1 activity on angiogenic cell mobilization and recovery of perfusion after limb ischemia. Circ Res. 2007; 101:1310-1318.
44. Zeoli A, Dentelli P, Rosso A, Togliatto G, Trombetta A, Damiano L, di Celle P F, Pegoraro L, Altruda F, Brizzi M F. Interleukin-3 promotes expansion of hemopoietic-derived CD45+ angiogenic cells and their arterial commitment via STAT5 activation. Blood. 2008; 112:350-361.
45. Musaro A, Barberi L. Isolation and culture of mouse satellite cells. Methods Mol. Biol. 2010; 633:101-111.
46. Dentelli P, Rosso A, Olgasi C, Camussi G, Brizzi M F. IL-3 is a novel target to interfere with tumor vasculature. Oncogene. 2011; 30:4930-4940.
47. Togliatto G, Trombetta A, Dentelli P, Rosso A, Brizzi M F. MIR221/MIR222-driven posttranscriptional regulation of P27KIP1 and P57KIP2 is crucial for high-glucose- and AGEmediated vascular cell damage. Diabetologia. 2011; 54:1930-1940.
48. Dentelli P, Rosso A, Orso F, Olgasi C, Taverna D, Brizzi M F. microRNA-222 controls neovascularization by regulating signal transducer and activator of transcription 5A expression. Arterioscler Thromb Vasc Biol. 2010; 30:1562-1568.
49. Delhanty P J, Neggers S J, van der Lely A J. Mechanisms in endocrinology: Ghrelin: the differences between acyl- and des-acyl ghrelin. Eur J Endocrinol 2012 November; 167(5):601-8.
50. Granata R, Settanni F, Julien M, Nano R, Togliatto G, Trombetta A, Gallo D, Piemonti L, Brizzi M F, Abribat T, van Der Lely A J, Ghigo E. Des-Acyl ghrelin fragments and analogues promote survival of pancreatic b-cells and human pancreatic islets and prevent diabetes in streptozotocin-treated rats. J. Med. Chem. 2012, 55, 2585-2596.

1. A method for protecting a subject against oxidative stress-induced damage, comprising administering an effective amount of unacylated ghrelin, a fragment thereof, an analog thereof and/or pharmaceutically acceptable salts thereof to the subject.

2. The method as defined in claim 1, wherein the oxidative stress-induced damage is oxidative stress-induced tissue damage.

3. The method as defined in claim 1, wherein the subject suffers from a neurodegenerative disease.

4. The method as defined in claim 1, wherein the subject suffers from atherosclerosis.

5. The method as defined in claim 1, wherein the subject suffers from peripheral artery disease.

6. The method as defined in claim 1, wherein the subject suffers from diabetes.

7. The method as defined in claim 1, for preventing an ischemia-reperfusion injury in the subject.

8. The method as defined claim 1, wherein the unacylated ghrelin is as set forth in SEQ ID NO: 1.

9. The method as defined in claim 1, wherein the fragment comprises amino acid residues 6 to 13 of SEQ ID NO: 1.

10. The method as defined in claim 1, wherein the fragment consists of an amino acid sequence selected from the group consisting of SEQ ID NO: 6, SEQ ID NO: 7 and SEQ ID NO: 8.

11. The method as defined in claim 1, wherein the fragment consists of amino acid sequence SEQ ID NO: 6.

12. The method as defined in claim 11, wherein the fragment is in a cyclized form.

13. The method as defined in claim 11, wherein the fragment comprises one or more of a linker moiety and a leader sequence.

14. The method as defined in claim 13, wherein the leader sequence is a mitochondrial leader sequence.

15. The method as defined in claim 1, for modulating cellular levels of superoxide-dismutase-2 (SOD-2).

16. The method as defined in claim 15, wherein the modulation of cellular levels of SOD-2 includes increasing cellular levels of SOD-2.

17. The method as defined in claim 15, wherein the modulation of cellular levels of SOD-2 includes increasing expression of SOD-2.

18. A method for protecting a population of cells against oxidative stress-induced damage, comprising contacting the population of cells with an effective amount of unacylated ghrelin, a fragment thereof, an analog thereof and/or pharmaceutically acceptable salts thereof.

19. The method as defined in claim 18, wherein the population of cells is an isolated population of cells.

20. The method as defined in claim 19, wherein the isolated population of cells in put in contact with the unacylated ghrelin, fragment thereof or analog thereof prior to transplantation in a subject.

21. The method as defined in claim 18, wherein cells of the population of cells are muscle tissue cells.

22. The method as defined in claim 21, wherein the muscle tissue cells are skeletal muscle tissue cells.

23. The method as defined in claim 18, wherein cells of the population of cells are ischemic cells.

24. The method as defined in claim 18, for modulating cellular levels of superoxide-dismutase-2 (SOD-2).

25. A method for modulating cellular levels of superoxide dismutase-2 (SOD-2) in a subject, comprising administering an effective amount of unacylated ghrelin, a fragment thereof, an analog thereof and/or pharmaceutically acceptable salts thereof to the subject.


SEQUENCE LISTING (30 sequences)

SEQ ID NO: 1 - protein, 28 aa, Homo sapiens
GSSFLSPEHQRVQQRKESKKPPAKLQPR

SEQ ID NO: 2 - protein, 14 aa, Homo sapiens
GSSFLSPEHQRVQQ

SEQ ID NO: 3 - protein, 18 aa, Homo sapiens
GSSFLSPEHQRVQQRKES

SEQ ID NO: 4 - protein, 5 aa, Homo sapiens
GSSFL

SEQ ID NO: 5 - protein, 12 aa, Homo sapiens
ESKKPPAKLQPR

SEQ ID NO: 6 - protein, 8 aa, Homo sapiens
SPEHQRVQ

SEQ ID NO: 7 - protein, 6 aa, Homo sapiens
EHQRVQ

SEQ ID NO: 8 - protein, 5 aa, Homo sapiens
EHQRV

SEQ ID NO: 9 - protein, 13 aa, Homo sapiens
SPEHQRVQQRKES

SEQ ID NO: 10 - protein, 4 aa, Homo sapiens
EHQR

SEQ ID NO: 11 - protein, 4 aa, Homo sapiens
HQRV

SEQ ID NO: 12 - protein, 8 aa, Homo sapiens
SPDHQRVQ

SEQ ID NO: 13 - protein, 8 aa, Homo sapiens
SPEHQKVQ

SEQ ID NO: 14 - protein, 8 aa, Homo sapiens
GPEHQRVQ

SEQ ID NO: 15 - protein, 8 aa, Homo sapiens
APEHQRVQ

SEQ ID NO: 16 - protein, 8 aa, Homo sapiens
SAEHQRVQ

SEQ ID NO: 17 - protein, 8 aa, Homo sapiens
SPAHQRVQ

SEQ ID NO: 18 - protein, 8 aa, Homo sapiens
SPEAQRVQ

SEQ ID NO: 19 - protein, 8 aa, Homo sapiens
SPEHARVQ

SEQ ID NO: 20 - protein, 8 aa, Homo sapiens
SPEHQAVQ

SEQ ID NO: 21 - protein, 8 aa, Homo sapiens
SPEHQRAQ

SEQ ID NO: 22 - protein, 8 aa, Homo sapiens
SPEHQRVA

SEQ ID NO: 23 - protein, 8 aa, Homo sapiens
  MOD_RES (1)..(1): ACETYLATION
SPEHQRVQ

SEQ ID NO: 24 - protein, 8 aa, Homo sapiens
  MOD_RES (1)..(1): ACETYLATION
SPEHQRVQ

SEQ ID NO: 25 - protein, 8 aa, Homo sapiens
  MISC_FEATURE (1)..(8): Cyclic
SPEHQRVQ

SEQ ID NO: 26 - protein, 8 aa, Homo sapiens
  MISC_FEATURE (3)..(6): Cyclic
SPEHQKVQ

SEQ ID NO: 27 - protein, 8 aa, Homo sapiens
  MOD_RES (1)..(1): ACETYLATION
SPEHQKVQ

SEQ ID NO: 28 - protein, 8 aa, Homo sapiens
  MOD_RES (1)..(1): ACETYLATION
SPEHQKVQ

SEQ ID NO: 29 - protein, 3 aa, Homo sapiens
HQR

SEQ ID NO: 30 - protein, 14 aa, Homo sapiens
QQVRQHEPSLFSSG